(19) United States
(12) Patent Application Publication
(10) Pub. No.: US 2024/0391873 A1
(43) Pub. Date: Nov. 28, 2024

(54) PROCESS FOR THE PREPARATION OF PEGYLATED ADRENOMEDULLIN, ITS INTERMEDIATES AND USE THEREOF

(71) Applicant: BAYER AKTIENGESELLSCHAFT, Leverkusen (DE)

(72) Inventors: Ralph O. Schönleber, Lupsingen (CH); Andreas Stadelmeier, Mühlheim (DE)

(21) Appl. No.: 18/683,911

(22) PCT Filed: Aug. 19, 2022
(86) PCT No.: PCT/EP2022/073150

§ 371 Date: Feb. 15, 2024

(30) Foreign Application Priority Data
Aug. 20, 2021 (EP) 21192392.5

(51) Int. Cl. C07D 207/416; C07C 315/04

(52) U.S. Cl. CPC C07D 207/416; C07C 315/04

(57) ABSTRACT

The invention refers to a process for the preparation of pegylated adrenomedullin according to formula (I), to intermediates used therein, and the use of the intermediates for the preparation of pegylated adrenomedullin according to formula (I).

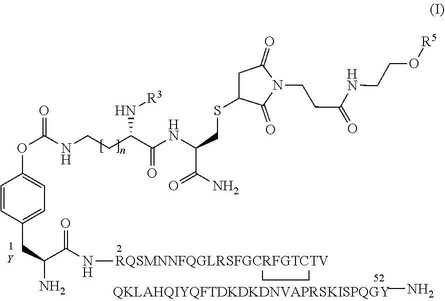

The invention refers to a process for the preparation of pegylated adrenomedullin according to formula (I)

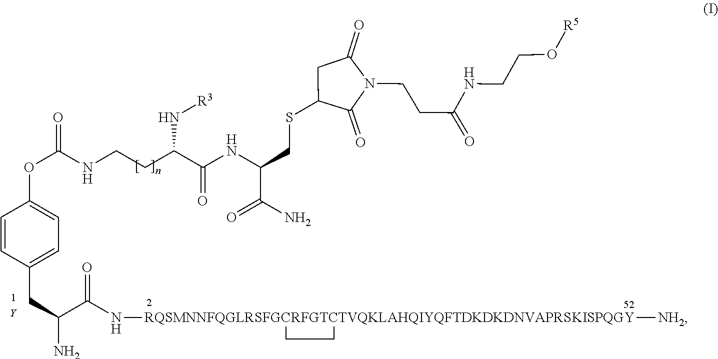

to intermediates used therein, and the use of the intermediates for the preparation of pegylated adrenomedullin according to formula (I).

Solid phase peptide synthesis (SPPS) is a well-established method in the industrial manufacture of peptide active pharmaceutical ingredients. In SPPS, a peptide anchored to an insoluble support—commonly a polymer resin—is assembled by the successive addition of the protected amino acid moieties constituting its sequence. Two types of protective groups are commonly used to suppress unwanted reactions during synthesis: “Temporary” protecting groups such as the base labile fluorenylmethyloxycarbonyl (Fmoc) on the alpha-amino group are employed to avoid multimerization and multiple insertions of the amino acid derivative to be added. They are removed from the growing peptide chain at the beginning of each elongation cycle. In contrast, amino acid side chain protecting groups are usually “permanent” in that the are maintained on the peptide chain throughout the entire synthesis. The skilled practitioner is well aware of a multitude of side chain protecting groups, which may be used in peptide synthesis. When one or more disulfide bridges are to be introduced into the molecule, specific protection schemes have been used (cf., e.g., Brochure “Solid Phase Peptide Synthesis Bachem—Pioneering Partner for Peptides”, published by Global Marketing, Bachem group, May 2020; Dekan et al., Angew. Chem. 2014, 126, 1-5; Postman and Albericio Eur. J. Org. Chem. 2014, 3519-3530, Patek and Lebl in: Peptides—Chemistry, Structure and Biology, Proceedings 13th APS, p. 146, June 20. 25 1993, ESCOM, Leiden 1994).

Poly(ethylene glycol)-conjugated peptides come along with new challenges with respect to engineering both their preparation and purification. Such challenges are related to such basic factors as polymer properties, and conjugation chemistry, as well as how these combine to alter the modified substance. For example, due to the sheer size of the PEG moiety compared to the peptide moiety, the properties of the PEGylated peptides may be dominated by the poly(ethylene glycol) (PEG) moiety. This may render the separation of PEGylated peptides differing only with respect to their peptide moiety very challenging.

PEGylated-proteins and PEGylated-peptides are an important class of therapeutics. Their manufacture typically encompasses covalent attachment of one or more PEG molecules to a native protein, which is then followed by purification steps. Pegylated prodrugs are known in the art. WO201364508A1 and WO2013064455A1 describe pegylated adrenomedullin according to formula (I),

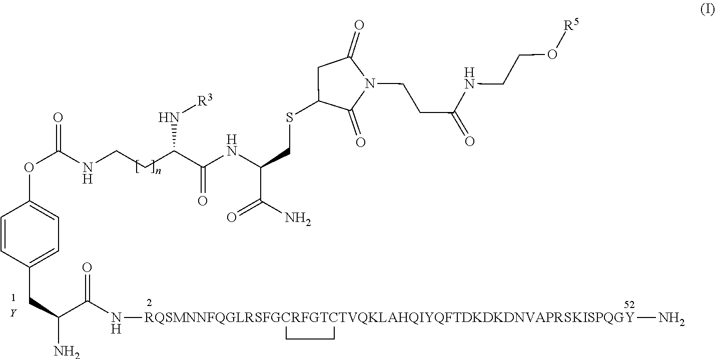

in which n represents the number 0, 1, 2 or 3, R³ represents hydrogen, methyl, ethyl, n-propyl or isopropyl, R⁵ represents linear or branched PEG 20 kDa to 80 kDa end-capped with a methoxy-group.

Formula (I) shows the uncharged compound. However, the skilled practitioner is well aware that peptides are amphiprotic molecules. Hence, depending on its environment, a compound according to formula (I) may be protonated at various positions, e.g. at its amino groups, may exhibit anionic groups, e.g. due to deprotonation of amino acid side chain carboxyl groups, and may bind to and/or form salts with various counter ions. This also applies for the remaining compounds depicted herein.

The compound according to formula (I) comprises the 52 amino acid peptide adrenomedullin and a pH sensitive (acid stabile) linker system. The manufacturing process of the compound according to formula according to formula (I) described in WO201364508A1 and WO2013064455A1 comprises the total synthesis of a polypeptide on solid support, a cyclization by formation of an internal disulfide bridge, attachment of the linker, cleavage from solid support and purification of the intermediate as well as PEGylation and purification of the drug substance. The prior art synthesis is depicted in scheme 1:

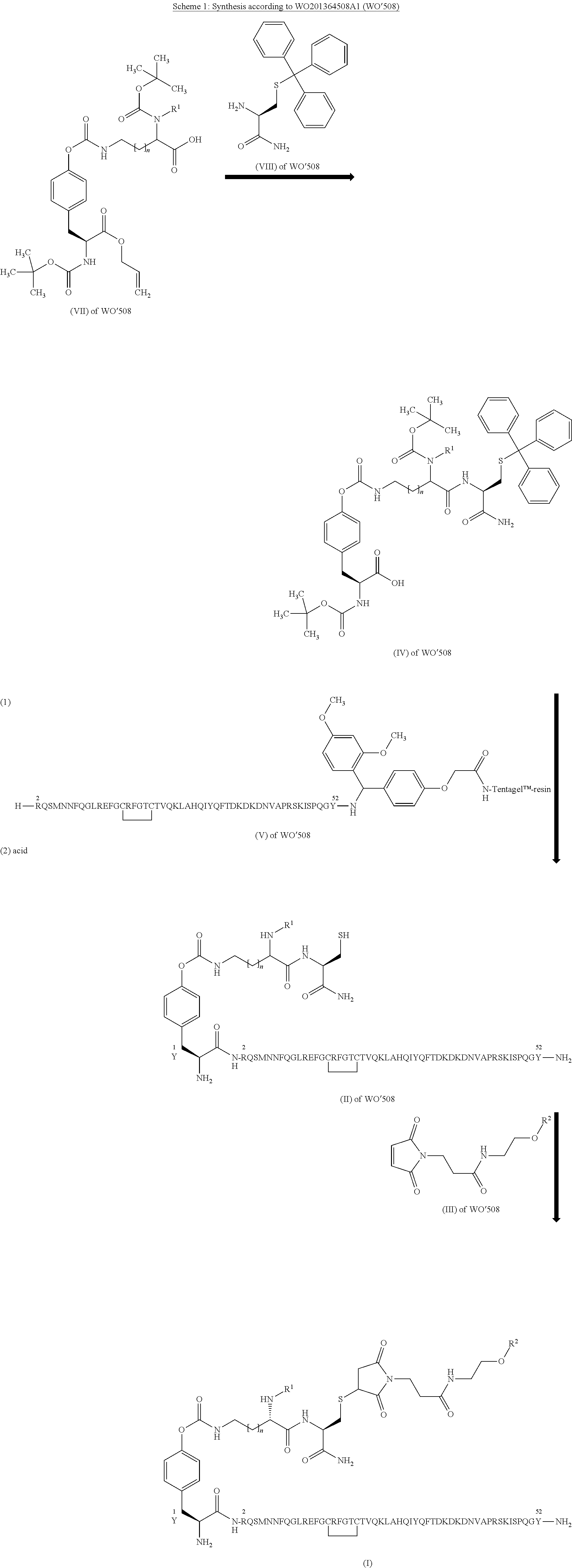

The above prior art process for the manufacture of the compound according to formula (I) exhibits several drawbacks. For example, on-resin formation of disulfide bonds tends to be less reproducible and is difficult to control. Further, because of the sheer size of the PEG moiety, it is extremely challenging to separate the compound of formula (I) from related substances, which exhibit minor variations in the peptide or linker part of the molecule. Hence, in an efficient and safe production method, the PEGylation reaction between intermediates according to formula (II) of WO201364508A1 and formula (III) of WO201364508A1 needs to avoid side product formation and is preferably carried out using well-controlled and pure educts. However, the present inventors found that the intermediate according to formula (II), which is formed upon cleavage from the solid support and concomitant cleavage of acid-labile protecting groups, is very challenging to produce and purify.

In order to meet the high quality and/or purity requirements for drug substances, it is mandatory to perform extensive chromatographic purification after solid phase synthesis of the adrenomedullin peptide chain, which comprises more than 50 amino acids. Unfortunately, this need for extensive purification conflicts with the fact that the stability of the intermediate (II) is limited. The linker's free sulfhydryl group is prone to undergo, e.g., disulfide reshuffling reactions, dimer formation, and oxidation during handling and analysis of the intermediate (II). Upon scale-up, prolonged hold and processing times have been found to accentuate these issues, thereby compromising product quality and yield.

The present inventors therefore devised a new synthesis strategy, which surprisingly allows to form the intermediate (II) reproducibly and in highly pure form immediately before the PEGylation reaction and without a need for a subsequent chromatographic purification step.

The present invention achieves these advantages by providing an improved linker structure, an improved linker peptide conjugate, and methods of producing and using the same. In particular, the linker and linker peptide conjugates of the present invention show improved stability due to the introduction of the safety-catch 4,4′-dimethylsulfinylbenzhydryl (Msbh) protecting group on the linker's sulfhydryl moiety. As a consequence, they are easy to produce and analyze and well suited for prolonged storage times. At the same time, the linker structure, the linker peptide conjugate, and the methods of the present invention allow to generate the intermediate (II) by controlled removal of the Msbh protecting group, which removal reaction is compatible with the subsequent PEGylation step without an intervening HPLC purification. As a further advantage, the compounds and methods of the present invention enable more efficient liquid phase methods of disulfide bond formation within the peptide chain. The compounds and methods of the present invention thus provide a robust and efficient manufacture process, which reproducibly delivers drug substance of high quality and allows seamless scale-up.

The invention refers to a process for the preparation of a compound according to formula (I)

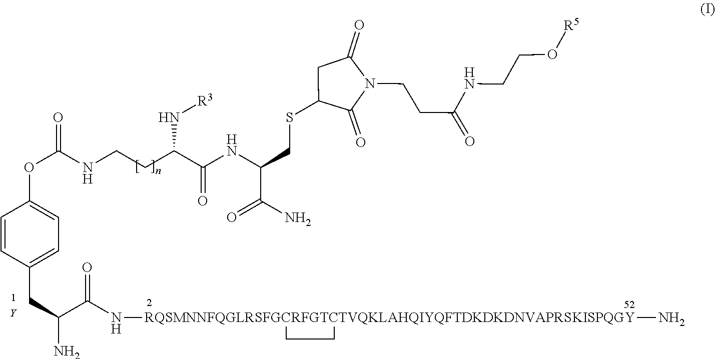

a hydrate thereof, solvate thereof, salt thereof, pharmaceutically acceptable salt thereof, or the solvates of salts thereof, comprising the following steps:
Step 1) Providing a compound according to formula (V)

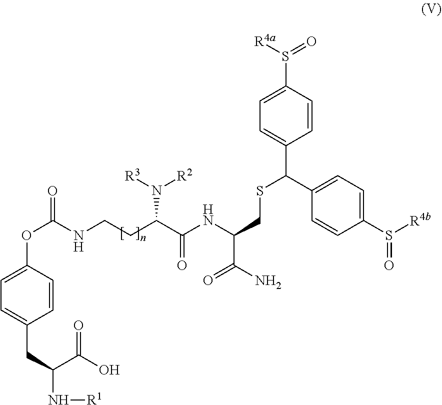

Step 2) Providing a compound according to formula (VIa) or according to formula (VIb)

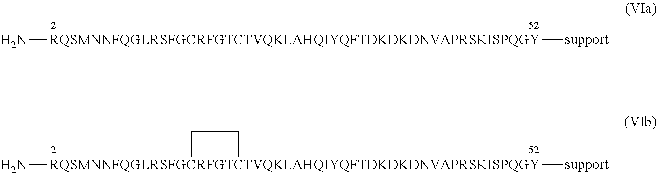

Step 3) Reacting the compound according to formula (V) with a compound according to formula (VIa) or (VIb), whereby a compound according to formula (VIIa) or (VIIb)

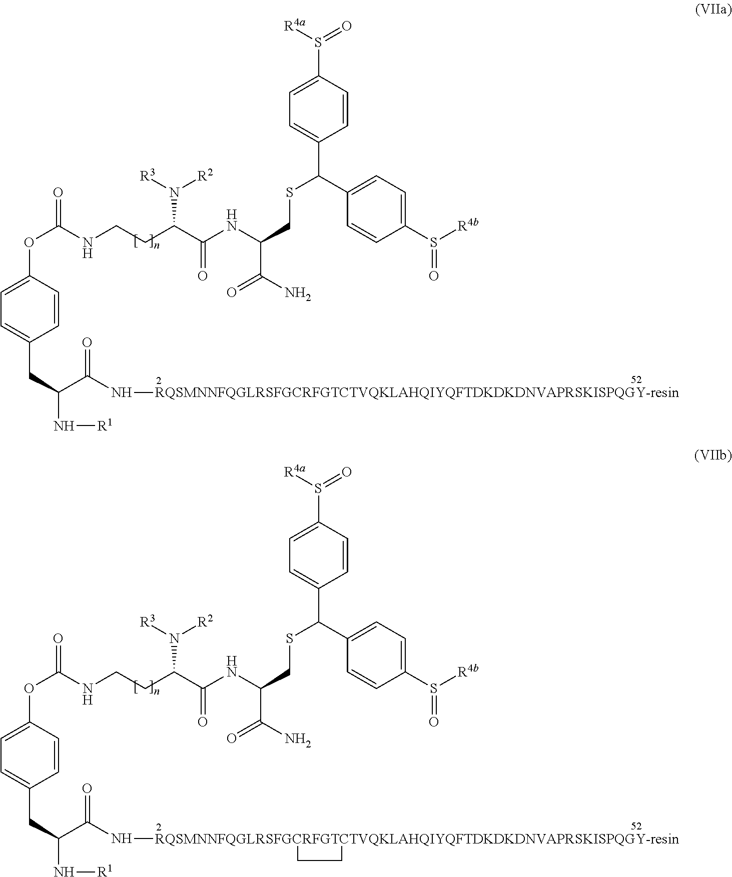

is obtained;
Step 4) Subjecting the compound according to formula (VIIa) or formula (VIIb) obtained in step 3) to an acid induced cleavage from the resin, whereby a compound according to formula (VIIIa) or (VIIIb)

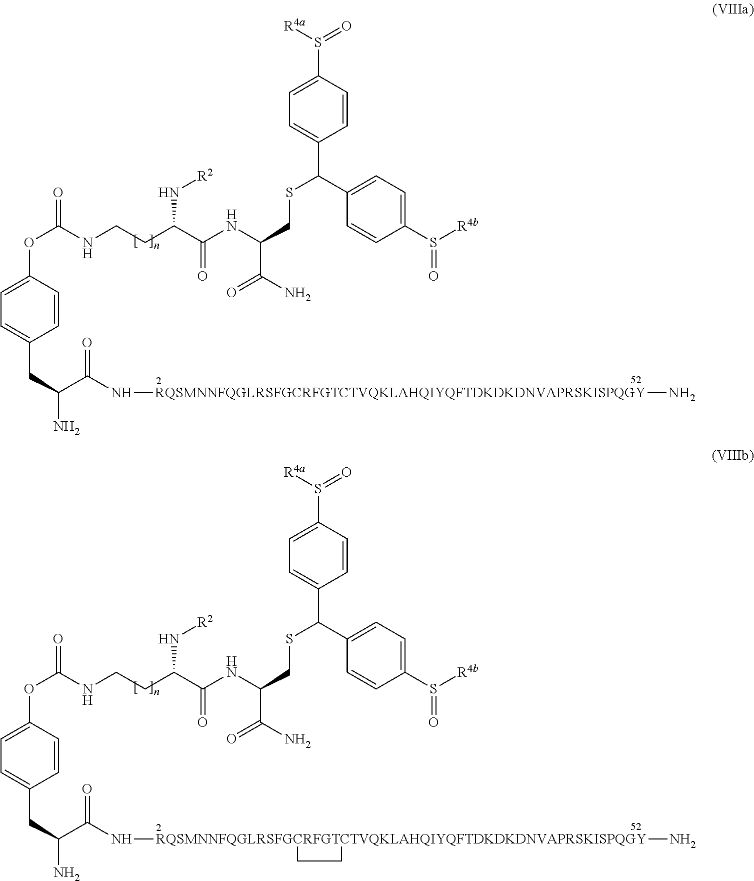

is obtained, wherein
if the compound according to formula (VIIa) or formula (VIIb) comprises as R¹ a (9H-fluoren-9-ylmethoxy)carbonyl group, said compound is subjected to a base-induced cleavage of the (9H-fluoren-9-ylmethoxy)carbonyl group and then to the acid induced cleavage from the resin, whereby a compound according to formula (VIIIa) or (VIIIb) is obtained;
Step 5) Optionally, if the product of step 4) is a compound according to formula (VIIIa), said compound is subjected to reaction with an oxidizing agent, whereby a compound according to formula (VIIIb) is obtained;
Step 6) Reacting the compound according to formula (VIIIb) obtained in step 4) or step 5) with a cleavage cocktail, whereby a compound according to formula (II)

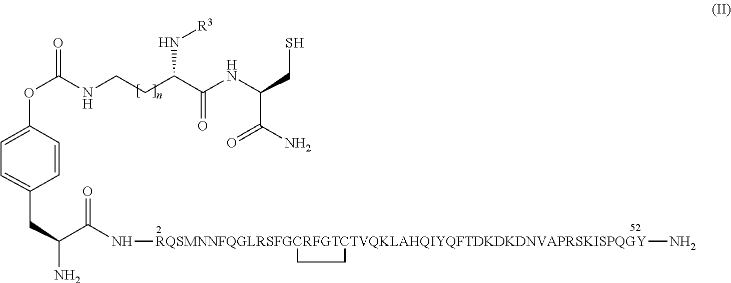

is obtained;
Step 7) Reacting the compound according to formula (II) obtained in step 6) with a compound according to the formula (IX)

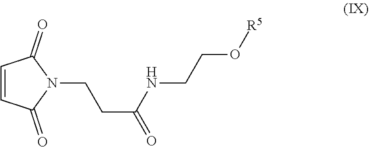

whereby the compound according to formula (I) is obtained;
in which n represents the number 0, 1 or 2,
R¹ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R⁴ represents methyl, ethyl, n-propyl, isopropyl or butyl,
R^{4b} represents methyl, ethyl, n-propyl, isopropyl or butyl,
R⁵ represents a linear or branched PEG 20 kDa to 80 kDa endcapped with a methoxy-group.

The process according to the invention is briefly depicted in scheme 2 below:

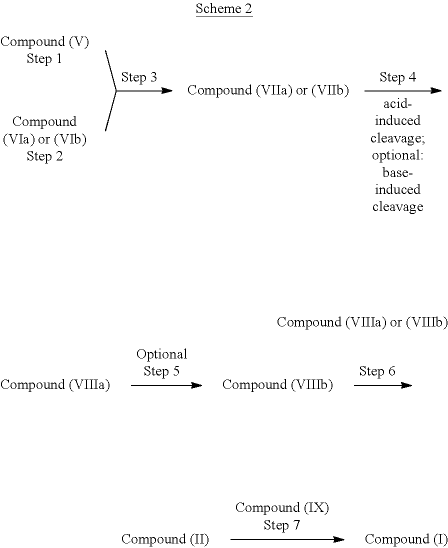

In one embodiment of the invention, the process comprises steps 1), 2), 3), 4), 5), 6) and 7). This is briefly disclosed in scheme 3 below:

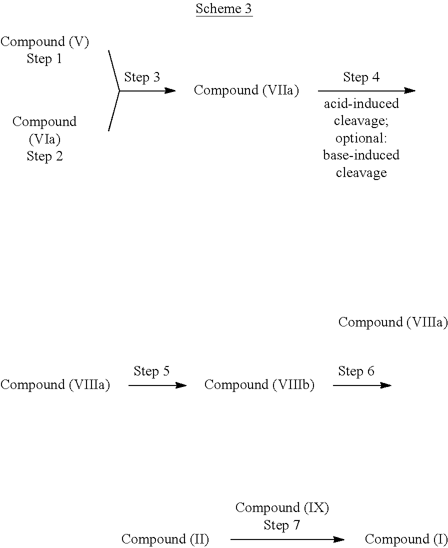

In one embodiment of the invention, the process comprises steps 1), 2), 3), 4), 6) and 7). This is briefly disclosed in scheme 4 below:

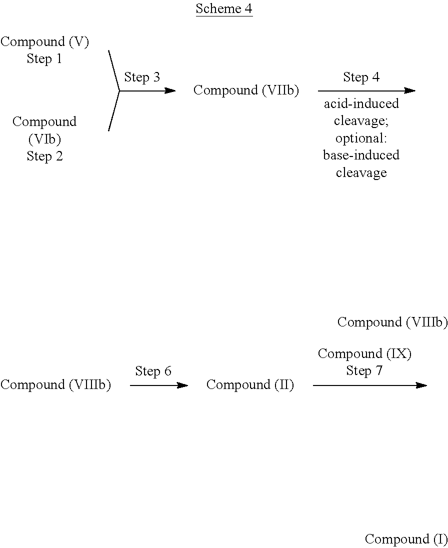

The process according to the invention provides for a compound according to formula (I)

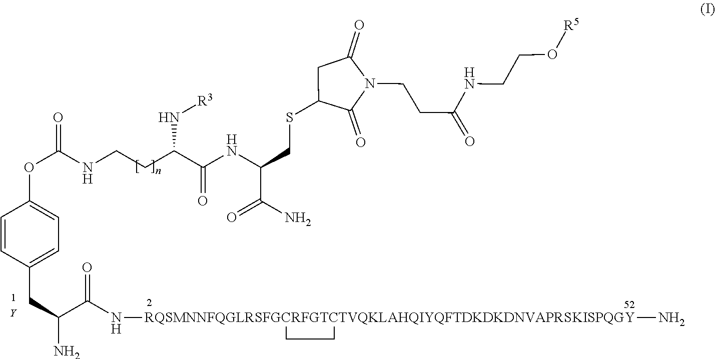

a hydrate thereof, solvate thereof, salt thereof, pharmaceutically acceptable salt thereof, or the solvates of salts thereof, in which
n represents the number 0, 1 or 2,
R³ represents hydrogen, methyl, ethyl, n-propyl or isopropyl,
R⁵ represents a linear or branched PEG 20 kDa to 80 kDa endcapped with a methoxy-group.

The terms “the compound of formula (I)” or “PEG-ADM” or “PEG-based prodrugs of ADM” or “Adrenomedullin Pegol” may be used as synonyms. The terms “the compound of formula (I)” or “PEG-ADM” or “PEG-based prodrug of ADM” or “Adrenomedullin Pegol” also include a hydrate thereof, solvate thereof, salt thereof, pharmaceutically acceptable salt thereof, or the solvates of salts thereof. Sometimes “PEG-ADM” is used as synonym for the compound according to formula (Ia).

Depending on their structure, the compounds according to the invention may exist in stereoisomeric forms (enantiomers, diastereomers). The invention therefore embraces the enantiomers or diastereomers and the particular mixtures thereof. The stereoisomerically homogeneous constituents can be isolated in a known manner from such mixtures of enantiomers and/or diastereomers.

When the compounds according to the invention can occur in tautomeric forms, the present invention embraces all tautomeric forms.

The term “compound” also includes a hydrate thereof, solvate thereof, salt thereof, pharmaceutically acceptable salt thereof, or the solvates of salts thereof.

The term “salt” is known in the art. The term “salt” may also include the terms “physiologically acceptable salts” or “pharmaceutically acceptable salts” or salts which are not suitable themselves for pharmaceutical applications. One embodiment refers to salts which are not suitable themselves for pharmaceutical application. One embodiment refers to salts which are not suitable themselves for pharmaceutical application, but, for example, can be used for the isolation or purification of the compounds according to the invention.

“Physiologically acceptable salts” or “pharmaceutically acceptable salts” of the compounds according to the invention include acid addition salts of mineral acids, carboxylic acids and sulfonic acids, for example salts of hydrochloric acid, hydrobromic acid, sulfuric acid, phosphoric acid, methane sulfonic acid, ethane sulfonic acid, toluene sulfonic acid, benzenesulfonic acid, naphthalene disulfonic acid, acetic acid, trifluoroacetic acid, propionic acid, lactic acid, tartaric acid, maleic acid, citric acid, fumaric acid, maleic acid and benzoic acid. “Physiologically acceptable salts” or “pharmaceutically acceptable salts” of the compounds according to the invention also include salts of customary bases, for example and with preference alkali metal salts (e.g. sodium and potassium salts), alkaline earth metal salts (e.g. calcium and magnesium salts) and ammonium salts derived from ammonia or organic amines having 1 to 16 carbon atoms, for example and with preference ethylamine, diethylamine, triethylamine, ethyl-diiso-propyl-amine, monoethanolamine, diethanolamine, triethanolamine, dicyclohexylamine, dimethylaminoethanol, procaine, dibenzylamine, N-methyl¬morpholine, arginine, lysine, ethylenediamine and N-methylpiperidine. Suitable pharmaceutically acceptable salts that can be used in the combination according to the invention are well known to those skilled in the art and include salts of inorganic acids, organic acids, inorganic bases, alkaline cations, alkaline earth cations and organic bases. In one embodiment the pharmaceutically acceptable salt can be selected from hydrochloric acid, hydrobromic acid, sulfuric acid, phosphoric acid, methane sulphonic acid, trifluoromethanesulfonic acid, benzenesulfonic acid, p-toluene sulfonic acid, 1-naphthalenesulfonic acid, 2-naphthalenesulfonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid, and mandelic acid acetate, benzoate, besylate, bromide, camsylate, carbonate, citrate, edisylate, estolate, fumarate, gluceptate, gluconate, glucuronate, hippurate, iodide, isethionate, lactate, lactobionate, malate, maleate, mesylate, methylsulfate, napsylate, nitrate, oxalate, pamoate, phosphate, stearate, succinate, sulfate, tartrate bitartrate, tosylate, calcium, diolamine, lithium, lysine, magnesium, meglumine, N-methylglucamine, olamine, potassium, tromethamine, tris(hydroxymethyl)aminomethane, benzenesulfonate, ethanesulfonate and zinc. In one embodiment the pharmaceutically acceptable salt can be selected from hydrochloride, sulfate, mesylate, tosylate, tartrate, citrate, benzenesulfonate, ethane sulfonate, maleate, and phosphate In the context of the invention, solvates refer to those forms of the compounds according to the invention which, in the solid or liquid state, form a complex by coordination with solvent molecules. Hydrates are a specific form of the solvates, in which the coordination is with water. In one embodiment, solvates in the context of the present invention are hydrates.

The compound according to formula (I) comprises a poly(ethylene glycol) (PEG) side chain endcapped with a methoxy group. This PEG moiety or PEG side chain is present in R⁵ in the formula (I). Theses side chains were already described in WO201364508A1. PEG is a polymer. The polymer can be of any molecular weight, and can be branched or unbranched. In some embodiments, the polyethylene glycol may have a branched structure. Branched polyethylene glycols are described, for example, in U.S. Pat. No. 5,643,575; Morpurgo et al., Appl. Biochem. Biotechnol. 56:59-72(1996); Vorobjev et al, Nucleosides Nucleotides 18:2745-2750 (1999); and Caliceti et al., Bioconjug. Chem. 10:638-646 (1999).

In some embodiments, the polyethylene glycol (PEG) may have a linear structure. In some embodiments, the polyethylene glycol may have a branched structure. In one embodiment of the invention, the PEG side chain is between about 1 kDa and about 100 kDa. In one embodiment, PEG is selected from 20 kDa, 25 kDa, 30 kDa, 35 kDa, 40 kDa, 45 kDa, 50 kDa, 55 kDa, 60 kDa, 65 kDa, 70 kDa, 75 kDa and 80 kDa, wherein the PEG is endcapped with a methoxy-group. According to one embodiment of the invention the PEG side chain is a linear or branched PEG 20 kDa to 80 kDa endcapped with a methoxy-group. In one embodiment of the invention, the PEG side chain is a linear or branched PEG 30 kDa to 60 kDa endcapped with a methoxy-group. In one embodiment of the invention, the PEG side chain is a linear or branched PEG 40 kDa endcapped with a methoxy-group.

Other sizes may be used, depending on the desired profile (e.g., the duration of sustained release desired, the effects, if any on biological activity, the ease in handling, the degree or lack of antigenicity and other known effects of the polyethylene glycol to a peptide or analog).

In one embodiment of the invention, the PEG side chain is a linear or branched PEG. In one embodiment of the invention, the PEG side chain is a linear PEG. In one embodiment of the invention, the PEG side chain is a branched PEG. Branched polyethylene glycols are described, for example, in U.S. Pat. No. 5,643,575; Morpurgo et al., Appl. Biochem. Biotechnol. 56:59-72(1996); Vorobjev et al, Nucleosides Nucleotides 18:2745-2750 (1999); and Caliceti et al., Bioconjug. Chem. 10:638-646 (1999).

The compound according to formula (I) comprises an adrenomedullin (ADM) sequence. Amino acids will be referred to interchangeably by either their full name (exemplified: alanine), 3-letter code (e.g. Ala), or 1-letter code (e.g. A). As far as the enantiomeric form is not expressly specified, L-amino acids are in general referred to. International Union of Pure and Applied Chemistry and International Union of Biochemistry: Nomenclature and Symbolism for Amino Acids and Peptides (Recommendations 1983). In: Pure & Appl. Chem. 56, Vol. 5, 1984, p. 595-624.

| Trivial Name | Symbol | One-letter Symbol |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “peptide” and “polypeptide” may be understood interchangeably. Unless indicated otherwise, peptide sequences are indicated herein starting with the N-terminus (left) and ending with the C-terminus (right). Substituents to the N-terminal amino group are indicated to the left of the sequence separated by a hyphen, and substituents to the C-terminal carboxyl group are indicated to the right of the sequence, separated by a hyphen. To stress that the N-terminus and C-terminus are not modified, they may be indicated as H- and —OH, respectively. For example, the notations H-GLA-OH, GLA, H-Gly-Leu-Ala-OH and Gly-Leu-Ala are all equivalent and refer to a tripeptide where the N-terminal amino group (“H”) and C-terminal carboxyl (“OH”) group are not modified. As a further example, the notation H-GLA-NH2 refers to a tripeptide having a unmodified N-terminal amino group and a carboxamide at the C-terminus. Substituents to amino acid side chains may be indicated in brackets to the right of the respective amino acid symbol. The analogous notation is used for amino acid derivatives. As used herein, an amino acid sequence (e.g. Arg-Ser-Lys aka. RSK) refers to both the unprotected peptide and derivatives with protected side chain moieties (e.g. Arg(pg1)-Ser(pg2)-Lys(pg3)). In order to expressly define the presence of protecting groups, their presence is indicated in brackets to the right of the respective amino acid symbol (e.g. Arg(pg1)-Ser(pg2)-Lys(pg3)).

The 52 amino acid sequence of ADM is as follows:

1 52
YRQSMNNFQGLRSFGCRFGTCTVQKLAHQIYQFTDKDKDNVAPRSKISPQGY

A disulfide bridge may be formed between Cys(16) and Cys(21).

“ADM(2-52)” is an 51 amino acid sub-sequence of ADM as follows:

2 52
RQSMNNFQGLRSFGCRFGTCTVQKLAHQIYQFTDKDKDNVAPRSKISPQGY

A disulfide bridge may be formed between Cys(16) and Cys(21). As used herein, the sulfhydryl side chains of an amino acid sequence will be considered to be —SH or —S-pg, where pg is a protecting group, unless specified otherwise. In order to expressly define the presence of a disulfide bridge in the molecule, the sequence may be indicated as:

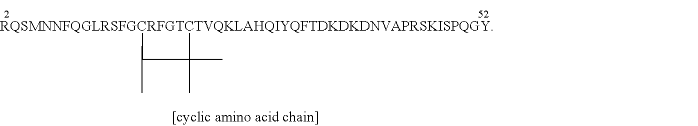

For example, the compound according to formula (VIa) comprises a linear ADM(2-52) sequence where both sulfhydryl side chains of the Cys moieties are —SH or S-pg, where pg is a protecting group.

2 52
H₂N-RQSMNNFQGLRSFGCRFGTCTVQKLAHQIYQFTDKDKDNVAPRSKISPQGY-support
(VIa)

The compound according to formula (VIb) comprises a cyclic ADM(2-52) sequence, in which a disulfide bridge is formed between Cys(16) and Cys(21), i.e. both sulfhydryl side chains of the Cys moieties are in oxidized state (—S—S—):

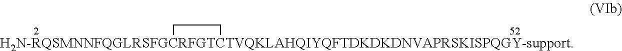

In one embodiment the process according to the invention provides for a compound according to formula (I), wherein
n represents the number 1,
R³ represents hydrogen, methyl, ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment the process according to the invention provides for a compound according to formula (I), wherein
n represents the number 1,
R³ represents hydrogen, methyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment the process according to the invention provides for a compound according to formula (I), wherein
n represents the number 1,
R³ represents hydrogen,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment the process according to the invention provides for a compound according to formula (I), wherein
n represents the number 1,
R³ represents hydrogen, methyl, ethyl,
R⁵ represents a linear or branched PEG 40 kDa endcapped with a methoxy-group,
a hydrate thereof, solvate thereof, salt thereof, pharmaceutically acceptable salt thereof, or the solvates of salts thereof.

In one embodiment the process according to the invention provides for a compound according to formula (I), wherein
n represents the number 1,
R³ represents hydrogen, methyl,
R⁵ represents a linear or branched PEG 40 kDa endcapped with a methoxy-group,
a hydrate thereof, solvate thereof, salt thereof, pharmaceutically acceptable salt thereof, or the solvates of salts thereof.

In one embodiment the process according to the invention provides for a compound according to formula (I), wherein
n represents the number 1,
R³ represents hydrogen,
R⁵ represents a linear or branched PEG 40 kDa endcapped with a methoxy-group,
a hydrate thereof, solvate thereof, salt thereof, pharmaceutically acceptable salt thereof, or the solvates of salts thereof.

In one embodiment the process according to the invention provides for a compound according to formula (Ia),

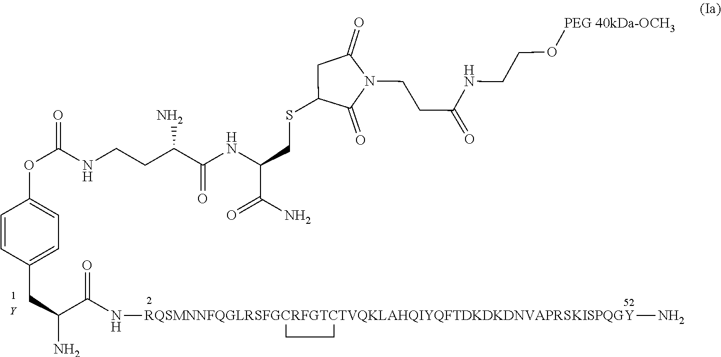

a hydrate thereof, solvate thereof, salt thereof, pharmaceutically acceptable salt thereof, or the solvates of salts thereof. The compound according to formula (Ia) is well described in detail in WO2013064508A1.

Its CAS number is 1432735-93-7. The International Nonproprietary Name (INN) of the compound according to formula (Ia) is “Adrenomedullin Pegol”.

In the following, further embodiments and aspects of the invention are described:

In step 1), a compound according to formula (V)

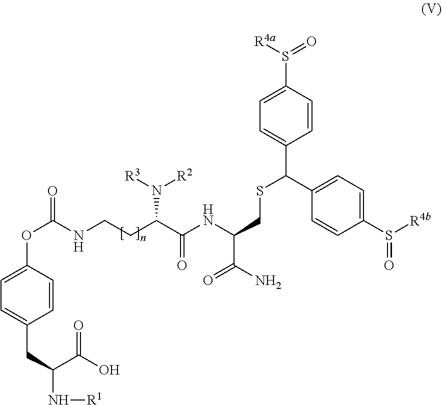

is provided,
in which
n represents the number 0, 1 or 2,
R¹ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R^{4a} represents methyl, ethyl, n-propyl, isopropyl or butyl,
R^{4b} represents methyl, ethyl, n-propyl, isopropyl or butyl.

This embodiment is briefly depicted in scheme 5:

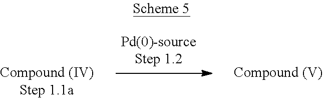

In one embodiment, the process for the preparation of the compound according to formula (I) comprises step 1.1.a), step 1.2), step 2), step 3), step 4), step 5), step 6) and step 7). In one embodiment, the process for the preparation comprises step 1.1.a), step 1.2), step 2), step 3), step 4), step 6) and step 7).

The compounds according to any one of formula (IV) and/or formula (V) can be defined as disclosed according to any one of the embodiments described herein. Step 1.2) can be conducted as disclosed according to any one of the embodiments described herein.

In one embodiment of step 1), the compound according to formula (V) provided in step 1) is prepared by the following process comprising the steps:
Step 1.1.a): Providing a compound according to formula (IV)

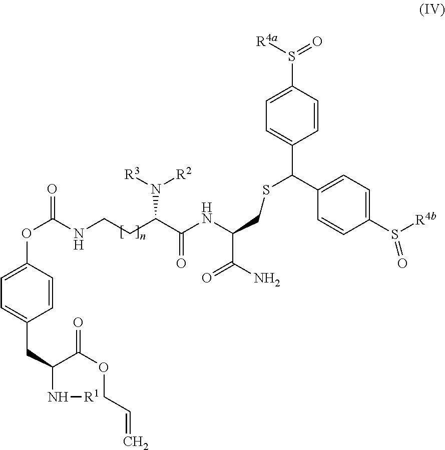

Step 1.2): Reacting the compound according to formula (IV) with a palladium(0) source and a nucleophile, whereby a compound according to formula (V) is obtained,
in which
n represents the number 0, 1 or 2,
R¹ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R^{4a} represents methyl, ethyl, n-propyl, isopropyl or butyl,
R^{4b} represents methyl, ethyl, n-propyl, isopropyl or butyl.

In one embodiment of step 1), the compound according to formula (V) provided in step 1) is prepared by the following process comprising the steps:
Step 1.1.a): Providing a compound according to formula (IV)
Step 1.2): Reacting the compound according to formula (IV) with a palladium(0) source and a nucleophile, whereby a compound according to formula (V) is obtained,
in which
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} and R^{4b} represent methyl or ethyl.

In one embodiment of step 1), the compound according to formula (V) provided in step 1) is prepared by the following process comprising the steps:
Step 1.1.a): Providing a compound according to formula (IV)
Step 1.2): Reacting the compound according to formula (IV) with a palladium(0) source and a nucleophile, whereby a compound according to formula (V) is obtained,
in which
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} represents methyl,
R^{4b} represents methyl.

Further embodiments of the compound according to formula (V) provided in step 1) or according to steps 1.1a) and 1.2) are provided below.

In one embodiment of step 1), the compound according to formula (V) provided in step 1) is prepared by the following process comprising the steps:
Step 1.1.b): Reacting a compound according to formula (X)

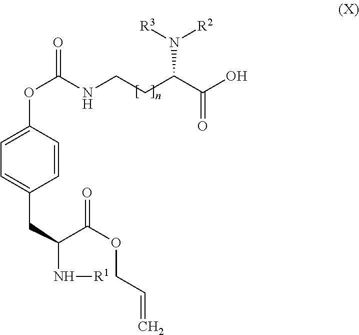

with a compound according to formula (III)

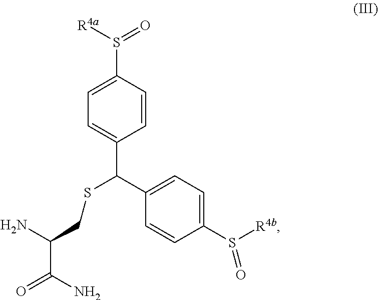

whereby a compound according to formula (IV) is obtained;
Step 1.2): Reacting the compound according to formula (IV) with a palladium(0) source and a nucleophile, whereby a compound according to formula (V) is obtained,
in which
n represents the number 0, 1 or 2,
R¹ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R^{4a} represents methyl, ethyl, n-propyl, isopropyl or butyl,
R^{4b} represents methyl, ethyl, n-propyl, isopropyl or butyl.

This embodiment is briefly depicted in scheme 6:

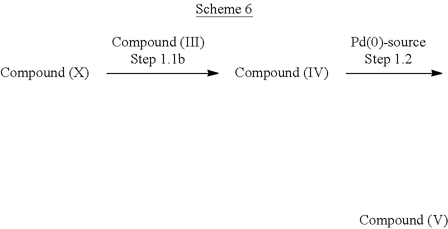

In one embodiment, the process for the preparation comprises step 1.1.b), step 1.2), step 2), step 3), step 4), step 5), step 6) and step 7). In one embodiment, the process for the preparation comprises step 1.1.b), step 1.2), step 2), step 3), step 4), step 6) and step 7).

The compounds according to any one of formula (X), formula (III), formula (IV) and/or formula (V) cm be defined as disclosed according to any one of the embodiments described herein. Step 1.2) can be conducted as disclosed according to any one of the embodiments described herein.

In one embodiment of step 1), the compound according to formula (V) provided in step 1) is prepared by the following process comprising the steps:
Step 1.1.b): Reacting a compound according to formula (X) with a compound according to formula (III), whereby a compound according to formula (IV) is obtained;
Step 1.2): Reacting the compound according to formula (IV) with a palladium(0) source and a nucleophile, whereby a compound according to formula (V) is obtained,
in which
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} and R^{4b} represent methyl or ethyl.

In one embodiment of step 1), the compound according to formula (V) provided in step 1) is prepared by the following process comprising the steps:
Step 1.1.b): Reacting a compound according to formula (X) with a compound according to formula (III), whereby a compound according to formula (IV) is obtained;
Step 1.2): Reacting the compound according to formula (IV) with a palladium(0) source and a nucleophile, whereby a compound according to formula (V) is obtained,
in which
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} represents methyl,
R^{4b} represents methyl.

In one embodiment, the compound according to formula (III) used in step 1.1.b) is 4,4′-dimethylsulfinylbenzhydryl (Msbh) protecting group (a compound according to formula (III-1))

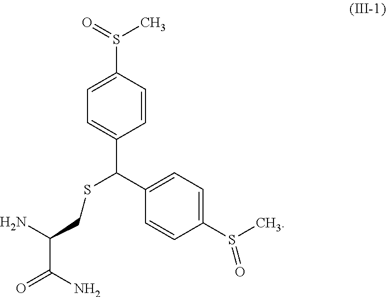 [SMILES: CS(=O)c1ccc(C(SC[C@H](N)C(N)=O)c2ccc(S(C)=O)cc2)cc1]

The compound according to formula (III-1) can be prepared as described in the Experimental Part, Example 3 and 4 below.

In one embodiment of step 1), the compound according to formula (V) provided in step 1) is prepared by the following process comprising the steps:
Step 1.1.b): Reacting a compound according to formula (X) with a compound according to formula (III-1), whereby a compound according to formula (IV) is obtained;
Step 1.2): Reacting the compound according to formula (IV) with a palladium(0) source and a nucleophile, whereby a compound according to formula (V) is obtained,
in which
n represents the number 0, 1 or 2,
R¹ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl.

In one embodiment of step 1), the compound according to formula (V) provided in step 1) is prepared by the following process comprising the steps:
Step 1.1.b): Reacting a compound according to formula (X) with a compound according to formula (III-1), whereby a compound according to formula (IV) is obtained;
Step 1.2): Reacting the compound according to formula (IV) with a palladium(0) source and a nucleophile, whereby a compound according to formula (V) is obtained,
in which
n represents the number 1,
R¹ represents tert-butyloxycarbonyl or (9H-fluoren-9-ylmethoxy)carbonyl,
R² represents tert-butyloxycarbonyl,
R³ represents hydrogen, methyl or ethyl.

In one embodiment of step 1), the compound according to formula (V) provided in step 1) is prepared by the following process comprising the steps:
Step 1.1.b): Reacting a compound according to formula (X) with a compound according to formula (III-1), whereby a compound according to formula (IV) is obtained;
Step 1.2): Reacting the compound according to formula (IV) with a palladium(0) source and a nucleophile, whereby a compound according to formula (V) is obtained,
in which
n represents the number 1,
R¹ represents tert-butyloxycarbonyl or (9H-fluoren-9-ylmethoxy)carbonyl,
R² represents tert-butyloxycarbonyl,
R³ represents hydrogen or methyl.

In one embodiment of step 1), the compound according to formula (V) provided in step 1) is prepared by the following process comprising the steps:
Step 1.1.b): Reacting a compound according to formula (X) with a compound according to formula (III-1), whereby a compound according to formula (IV) is obtained;
Step 1.2): Reacting the compound according to formula (IV) with a palladium(0) source and a nucleophile, whereby a compound according to formula (V) is obtained,
in which
n represents the number 1,
R¹ represents tert-butyloxycarbonyl or (9H-fluoren-9-ylmethoxy)carbonyl,
R² represents tert-butyloxycarbonyl,
R³ represents hydrogen.

Further embodiments of the compound according to formula (V) provided in step 1) or according to steps 1.1a) and 1.2) are provided below.

In one embodiment, step 1.1.b) is carried out in an inert solvent selected from halohydrocarbons, ethers or mixtures thereof. In one embodiment, step 1.1.b) is carried out in an inert solvent selected from N,N-dimethylformamide (DMF), 2-methyltetrahydrofuran (MeTHF), tetrahydrofuran (THF), trichloromethane, 1,2-dichloroethane, dioxane, 1,2-dimethoxyethane, acetone, dimethylacetamide, 2-butanone, acetonitrile, simethyl sulphoxide (DMSO), ethyl acetate, 1,3-dioxolane, toluene, N-butyl pyrrolidone, dimethyl isosorbide, gamma-valerolactone, 2,5,7,10-tetraoxaundecane (TOU), dihydrolevo-glucosenone (Cyrene), tetrahydropyran (THP), N-formyl-morpholine (NFM), acetonitrile, propylene carbonate (PC), anisole and mixtures thereof.

In one embodiment, step 1.1.b) is carried out at a temperature between −5° C. and 50° C. In one embodiment, step 1.1.b) is carried out at a temperature between 0° C. and 40° C., In one embodiment, step 1.1.b) is carried out at a temperature between 0° C. and 35° C.

In one embodiment, step 1.1.b) is carried out in the presence of a coupling reagent. In one embodiment, the coupling reagent in step 1.1.b) is selected from N,N′-diethyl-carbodiimide, N,N′-dipropyl-carbodiimide, N,N′-diisopropyl carbodiimide, N,N′-dicyclohexyl-carbodiimide, N-(3-dimethylaminoisopropyl)-N′-ethyl-carbodiimide hydrochloride (EDC), N-cyclohexylcarbodiimide-N′-propyloxymethyl-polystyrene (PS-carbodiimide), carbonyldiimidazole, 2-ethyl-5-phenyl-1,2-oxazolium 3-sulphate or 2-tert-butyl-5-methylisoxazolium perchlorate, 2-ethoxy-1-ethoxycarbonyl-1,2-dihydroquinoline, propanephosphonic anhydride, isobutyl, chloroformate, bis-(2-oxo-3-oxazolidinyl)phosphoryl chloride, benzotriazolyloxytri(dimethylamino)phospho¬nium hexafluorophosphate, O-(benzo-triazol-1-yl)-N,N,N′,N′-tetramethyluronium hexafluorophosphate (HBTU), benzotriazol-1-yl-N-tetramethyl-uroniumtetrafluoroborate (TBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU), O-(7-Azabenzotriazole-1-yl)-N,N,N′,N′-tetramethyluronium tetrafluoroborate (TATU), ethyl cyanohydroxyiminoacetate (oxyma), 1-hydroxybenzotriazole (HOBt) in combination with an carbodiimid, benzotriazol-1-yloxytris(dimethylamino)phosphonitnhexafluorophosphate (BOP), N-hydroxysuccinimide in combination with an carbodiimide, benzotriazol-1-yloxytris(pyrrolidino)phosphonium hexafluorophosphate (PYBOP), ((1-Cyano-2-ethoxy-2-oxoethylidenaminooxy)dimethylamino-morpholino-carbenium-hexafluorophosphat) (COMU), (3-(diethoxyphosphoryloxy)-1,2,3-benzotriazin-4-one) (DEPBT), [Ethyl-cyano(hydroxyimino)acetato-O2]tri-1-pyrrolidinylphosphonium hexafluorophosphate (PyOxim), and mixtures thereof.

In one embodiment, step 1.1.b) is carried out in the presence of a base. In one embodiment, step 1.1.b) is carried out in the presence of a base selected from alkalimetal carbonates, organic bases and mixtures thereof. In one embodiment, step 1.1.b) is carried out in the presence of a base selected from sodium carbonate, potassium carbonate, sodium hydrogencarbonate, potassium hydrogencarbonate, triethylamine, triethylamine, N-methylmorpholine, N-methylpiperidine, 4-dimethyl¬amino¬pyridine N,N-diisopropylethylamine, potassium phosphate solution, N,N-diisopropylethylamine (DIPEA, Hünig's base), and mixtures thereof.

A further aspect is the use of the compound according to formula (III-1) for the preparation of the compound according to formula (IV). A further aspect is the use of the compound according to formula (III-1) for the preparation of the compound according to formula (V). A further aspect is the use of the compound according to formula (III-1) for the preparation of the compound according to formula (VIIa) or (VIIb). A further aspect is the use of the compound according to formula (III-1) for the preparation of the compound according to formula (VIIIa) or (VIIIb). A further aspect is the use of the compound according to formula (III-1) for the preparation of the compound according to formula (I). A further aspect is the use of the compound according to formula (III-1) for the preparation of the compound according to formula (Ia).

In one embodiment, the palladium source in step 1.2) is selected from the group consisting of tetrakis(triphenylphosphine)palladium(0) ((Pd(PPh₃)₄)), palladium(II)bis(triphenylphosphine) dichloride (PdCl₂(Ph₃P)₂), palladium(II)-acetate (Pd(OAc)₂), palladium on charcoal (Pd/C) and mixtures thereof. In one embodiment, the palladium(0) source in step 1.2) is tetrakis(triphenylphosphine)palladium(0) ((Pd(PPh₃))).

In one embodiment, the nucleophile in step 1.2) is selected from formic acid, acetic acid, triethyl silane, pyrrolidine, morpholine, dimedone, dimethyl barbituric acid, dimethylamine borane complex and mixtures thereof.

In step 2), a compound according to formula (VIa) or according to formula (VIb)

2 52
H₂N-RQSMNNFQGLRSFGCRFGTCTVQKLAHQIYQFTDKDKDNVAPRSKISPQGY-support
(VIa)

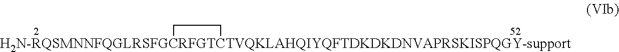

is provided. The compound according to formula (VIa) or according to formula (VIb) comprises a ADM(2-52) peptide chain linked to a support.

In one embodiment of step 2), a compound according to formula (VIa) is provided. In one embodiment of step 2), a compound according to formula (VIb) is provided.

In one embodiment of step 2), the support in formula (VIa) or the support in formula (VIb) is selected from the group consisting of polystyrene, functionalized polystyrene, carrying end-groups that allow for the attachment of carboxylic acids via an amide bond, Rink linker, tricyclic amide linker, xanthenyl linker, polyethylene glycol, polyethylene glycol-polystyrene. Further embodiments of possible supports are Rink-Amide type polystyrene, Ramage resin and/or Tentagel™.

In one embodiment of step 2), some amino acid side chains of the compound according to formula (VIa) or (VIb) bear a protecting group (pg), i.e. at least one reactive moiety located in an amino acid side chain of the compound according to formula (VIa) or (VIb) is modified by a protecting group. In one embodiment of step 2), all carboxyl, amino and sulfhydryl moieties, which are located in an amino acid side chain bear protecting groups.

The at least one or more side chain protecting groups can be positioned in the ADM(2-52) amino acid sequence as depicted below in formula (VIa-1) or (VIb-1):

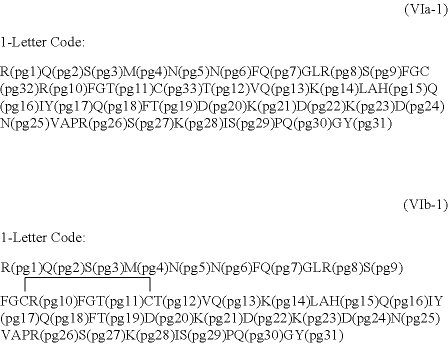

In one embodiment, each protecting group is independently missing or is selected independently from 2,2,5,7,8-pentamethylchroman-6-sulfonyl (Pmc), 2,2,4,6,7-pentamethyldihydrobenzofuran-5-sulfonyl (Pbf), 4,4′-dimethoxytrityl (DMT), 4-methoxy-2,3,6-trimethylbenzenesulphonyl (Mtr), 4-methyltrityl (Mtt), xanthenyl (Xan), O-3-methylpent-3-yl (OMpe), β-3-methylpent-3-yl (Mpe), allyl ester (OAll), 4-{-[1-(4,4-dimethyl-2,6-dioxocyclohexylidene)-3-methylbutyl]amino}benzyl ester (ODmab), tert-butyloxycarbonyl (Boc), allyloxycarbonyl (Alloc), 2-(1′-adamantyl)-2-propyloxycarbonyl (Adpoc), 1-(4,4-dimethyl-2,6-dioxocyclohexylidene)-3-methylbutyl (Dde), 1-(4,4-dimethyl-2,6-dioxocyclohex-1-ylidene)-3-methylbutyl (ivDde), benzyloxycarbonyl (Z), 9-fluorenylmethoxycarbonyl(Fmoc), tert-butyl (tBu), triphenylmethyl (Trt), benzyl (Bzl), diphenylmethyl (Dpm), acetamidomethyl (Acm), 2-{[(4-methoxytritylthio)oxy]methyl}benzoate (Mob), p-methoxyphenyldiphenylmethyl(Mmt), 4-methylbenzyl (Mbzl), tert-butylthio (StBu) groups, oxazolidine groups formed between the side chain and the alpha-amino group of Ser, Thr and Cys, and thiazolidine groups formed between the side chain and the alpha-amino group of Ser, Thr and Cys.

The protecting groups (pg) pg1 through pg31 are chosen independently of each other and may be the same or different. Some of the protecting groups, in particular pg4, may be missing. The protecting groups pg1 through pg31 may be chosen among the protecting groups known in the field. Examples of protecting groups comprise the 2,2,5,7,8-Pentamethylchroman-6-sulfonyl (Pmc), 2,2,4,6,7-Pentamethyldihydrobenzofuran-5-sulfonyl (Pbf), 4,4′-Dimethoxytrityl (DMT), 4-Methoxy-2,3,6-trimethylbenzenesulphonyl (Mtr), 4-methyltrityl (Mtt), xanthenyl (Xan), O-3-methylpent-3-yl (OMpe), allyl ester (OAll), 4-{-[1-(4,4-dimethyl-2,6-dioxocyclohexylidene)-3-methylbutyl]amino}benzyl ester (ODmab), tert-butyloxycarbonyl (Boc), Allyloxycarbonyl (Alloc), 2-(1′-Adamantyl)-2-propyloxycarbonyl (Adpoc), 1-(4,4-Dimethyl-2,6-dioxocyclohexylidene)-3-methylbutyl (Dde), 1-(4,4-dimethyl-2,6-dioxocyclohex-1-ylidene)-3-methylbutyl (ivDde), Benzyloxycarbonyl (Z), 9-Fluorenylmethoxycarbonyl (Fmoc), tert-Butyl (tBu), Triphenylmethyl (=Trityl) (Trt), benzyl (BA), diphenylmethyl (Dpm), Acetamidomethyl (Acm), 2-{[(4-methoxytritylthio)oxy]methyl}benzoate (Mob), p-methoxyphenyldiphenylmethyl (Mmt), 4-Methylbenzyl (Mbzl), and tert-Butylthio (StBu) groups Further examples of protective groups comprise oxazolidine and thiazolidine groups formed between the side chain and the alpha-amino group of Ser, Thr and Cys. These protecting groups may be introduced into the peptide sequence by coupling of the corresponding Fmoc dipeptide of the antecedent amino acid with the oxazolidine or thiazolidine derivative of the following amino acid. Such dipeptides are often referred to as pseudoproline dipeptides, are readily available, and may be used to prevent on-resin aggregation. In one embodiment, at least one protecting group selected from pg3, pg9, pg11, pg12, pg19, pg27, and pg29 may be an oxazolidine, preferably with two methyl substituents at the 2-position of the oxazolidine ring ((Psi(Me,Me)pro)). In another embodiment, at least two protecting groups selected from pg3, pg9, pg11, pg12, pg19, pg27, and pg29 may be an oxazolidine, preferably with two methyl substituents at the 2-position of the oxazolidine ring ((Psi(Me,Me)pro)). In another embodiment, at least two protecting groups may be an oxazolidine, preferably with two methyl substituents at the 2-position of the oxazolidine ring ((Psi(Me,Me)pro)), and the positions of the oxazolidines are selected from the group consisting of pg3 and pg9; pg3 and pg11; pg3 and pg12; pg3 and pg19; pg3 and pg27; pg3 and pg29; pg9 and pg11; pg9 and pg12; pg9 and pg19; pg9 and pg27; pg9 and pg29; pg11 and pg12; pg11 and pg19; pg11 and pg27; pg11 and pg29; pg12 and pg19; pg12 and pg27; pg12 and pg29, pg19 and pg27; pg19 and pg29; and pg27 and pg29. In another embodiment, at least two protecting groups may be an oxazolidine preferably with two methyl substituents at the 2-position of the oxazolidine ring ((Psi(Me,Me)pro)), ad the positions of the oxazolidines are selected from the group consisting of pg3 and pg9; pg3 and pg11; pg3 and pg19; pg9 and pg11; pg9 and pg19; and pg11 and pg19.

The compound according to formula (VIa) or (VIb) may be assembled using at least one dipeptide selected from the group consisting of Fmoc-Gln(Trt)-Ser(Psi(Me,Me)pro)-OH, Fmoc-Arg(Pbf)-Ser(Psi(Me,Me)pro)-OH, Fmoc-Gly-Thr(Psi(Me,Me)pro)-OH, Fmoc-Phe-Thr(Psi(Me,Me)pro)-OH, and Fmoc-Ile-Ser (Psi(Me,Me)pro)-OH. In some embodiments of the compound according to formula (VIa), at least one protecting group selected from pg32 and pg33 may be an thiazolidine, preferably with two methyl substituents at the 2-position of the thiazolidine ring ((Psi(Me,Me)pro)). In some embodiments, the compound according to formula (VIa) may be assembled using at least one dipeptide selected from the group consisting of Fmoc-Gly-Cys(Psi(Me,Me)pro)-OH and Fmoc-Thr(tBu)-Cys(Psi(Me,Me)pro)-OH.

In some embodiments, the protecting groups pg1 through pg33 are base resistant, i.e. they are essentially resistant to conditions, under which Fmoc cleavage from a peptide is performed Preferably, the protecting groups pg1 through pg3 and pg5 though pg33 are base resistant and acid labile, i.e. cleavable upon treatment with trifluoroacetic acid. In some embodiments, at least the protecting groups pg1 through pg3 and pg5 though pg31 are base resistant and acid labile. In some embodiments, the compound according to formula (VIa) or (VIb) comprises at least one oxazolidine protecting group of a Ser or Thr side chain, and all side chains of Arg, Gin, Ser, Asn, Gin, Cys, Thr, Lys, His, Tyr and Asp carry side chain protecting groups. In some embodiments, the compound according to formula (VIa) comprises two oxazolidine protecting groups of a Ser or Thr side chain, and all side chains of Arg, Gln, Ser, Asn, Gin, Cys, Thr, Lys, His, Tyr and Asp carry side chain protecting groups, preferably acid labile side chain protecting groups In some embodiments, the compound according to formula (VIa) or (VIb) comprises at least one, preferably two, oxazolidine protecting group(s) of a Ser or Thr side chain, and all other side chains of Arg Gln, Ser, Asn, Gin, Cys, Thr, Lys, His, Tyr and Asp carry side chain protecting groups, which are independently selected from the group consisting of: 2,2,4,6,7-pentamethyldihydrobenzofuran-5-sulfonyl (Pbf), triphenylmethyl (Trt), tert. butyl (tBu), diphenylmethyl (Dpm), t-butoxycarbonyl (Boc), and O-3-methylpent-3-yl (OMpe).

The compound according to formula (VIa) or (VIb) may be provided in any conceivable way. For example, the compound may be provided by solid phase peptide synthesis (SPPS), preferably by Fmoc-SPPS. This may comprise the subsequent coupling of single amino acid derivatives and, optionally, dipeptides such as Fmoc-pseudoproline dipeptides. Other possibilities of synthesis comprise the coupling of larger fragments, preferably fragments with an N-terminal glycine or proline residue. Such fragments may be synthesized previously by either liquid phase peptide synthesis or by solid phase peptide synthesis.

A compound according to formula (VIb) may be obtained by oxidation of the protected or unprotected sulfhydryl-groups on the resin, e.g. using iodine as an oxidizing agent. The corresponding methods are well known and readily available to the skilled practitioner. The compound according to formula (VIa or according to formula (VIb) is also known in the art and was already described in WO201364508A1. The compound according to formula (VIa) or according to formula (VIb) can be prepared as described in the Experimental Part, Example 7a or Example 7b below.

In step 3), the compound according to formula (V) is reacted with a compound according to formula (VIa) or (VIb), whereby a compound according to formula (VIIa) or (VIIb)

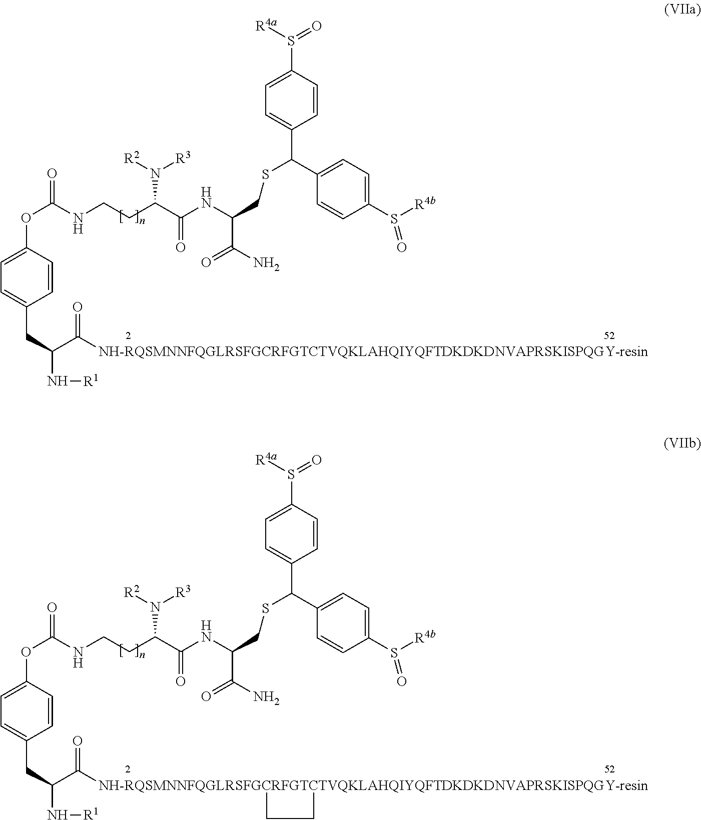

is obtained, in which
n represents the number 0, 1 or 2,
R⁵ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R^{4a} represents methyl, ethyl, n-propyl, isopropyl or butyl,
R^{4b} represents methyl, ethyl, n-propyl, isopropyl or butyl.

In one embodiment the compound according to formula (V) is reacted with a compound according to formula (VIa) or (VIb), whereby a compound according to formula (VIIa) or (VIIb) is obtained,
in which
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} and R^{4b} represent methyl or ethyl.

In one embodiment the compound according to formula (V) is reacted with a compound according to formula (VIa) or (VIb), whereby a compound according to formula (VIIa) or (VIIb) is obtained,
in which
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} represents methyl,
R^{4b} represents methyl.

The compounds according to any one of formula (VIIa) and/or formula (VIIb) can be defined as disclosed according to any one of the embodiments described herein.

In one embodiment of step 3), the resin in the compound according to formula (VIIa) or (VIIb) is selected from Ramage aka Tricyclic amide linker resin, Rink amide resin, Xanthenyl resin, Sieber resin and mixtures thereof.

In one embodiment, step 3) is conducted in an inert solvent. In one embodiment, the inert solvent in step 3) is selected from halohydrocarbons, ethers and mixtures thereof. In one embodiment, the inert solvent in step 3) is selected from 2-butanone, acetone, dimethylformamide, dimethylacetamide, 2-butanone, acetonitrile, N-methyl-2-pyrrolidon (NMP), dimethyl carbonate (DMC), dichloromethane, trichloromethane, 1,2-dichloroethane, dioxane, tetrahydrofuran, 1,2-dimethoxyethane, acetone, dimethylformamide and mixtures thereof.

In one embodiment, step 3) is conducted in the presence of a coupling reagent. In one embodiment, the coupling reagent in step 3) is selected from N,N′-diethyl-carbodiimide, N,N′-dipropyl-carbodiimide, N,N′-diisopropyl carbodiimide, N,N′-dicyclohexyl-carbodiimide, N-(3-dimethylaminoisopropyl)-N-ethyl-carbodiimide hydrochloride (EDC), N-cyclohexylcarbodiimide-N′-propyloxymethyl-polystyrene (PS-carbodiimide), carbonyldiimidazole, 2-ethyl-5-phenyl-1,2-oxazolium 3-sulphate or 2-tert-butyl-5-methylisoxazolium perchlorate, 2-ethoxy-1-ethoxycarbonyl-1,2-dihydroquinoline, propanephosphonic anhydride, isobutyl, chloroformate, bis-(2-oxo-3-oxazolidinyl)phosphoryl chloride, benzotriazolyloxytri(dimethylamino)phospho-nium hexafluorophosphate, O-(benzo-triazol-1-yl)-N,N,N′,N′-tetramethyluronium hexafluorophosphate (HBTU), benzotriazol-1-yl-N-tetramethyl-uronium tetrafluoroborate (TBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU), O-(7-Azabenzotriazole-1-yl)-N,N,N′,N′-tetramethyluronium tetrafluoroborate (TATU), ethyl cyanohydroxyiminoacetate (oxyma), 1-hydroxybenzotriazole (HOBt) in combination with an carbodiimid, benzotriazol-1-yloxytris(dimethylamino)phosphoniumhexafluorophosphate (BOP), N-hydroxysuccinimide in combination with an carbodiimide, benzotriazol-1-yloxytris(pyrrolidino)phosphonium hexafluorophosphate (PYBOP), ((1-Cyano-2-ethoxy-2-oxoethylidenaminooxy)dimethylamino-morpholino-carbenium-hexafluorophosphat) (COMU), (3-(diethoxyphosphoryloxy)-1,2,3-benzotriazin-4-one) (DEPBT), [Ethyl cyano(hydroxyimino)acetato-O2]tri-1-pyrrolidinylphosphonium hexafluorophosphate (PyOxim), and mixtures thereof.

In one embodiment, step 3) is carried out in the presence of a base. In one embodiment, the base in step 3) is selected from alkalimetal carbonates, organic bases and mixtures thereof. In one embodiment, the base in step 3) is selected from alkylamines, dialkylamines, trialkylamines, and mixtures thereof. In one embodiment, the base in step 3) is selected from sodium carbonate, potassium carbonate, sodium hydrogencarbonate, potassium hydrogencarbonate, triethylamine, N-methyl-morpholine, N-methylpiperidine, 4-dimethylaminopyridine, N,N-diisopropylethylamine and mixtures thereof.

In one embodiment, the base treatment in step 3) is carried out at a temperature between 0° C. to 70° C. In one embodiment, the base treatment in step 3) is carried out at a temperature between 20° C. to 70° C. In one embodiment, the base treatment in step 3) is carried out at a temperature between 15° C. to 35° C.

In one embodiment, the base treatment in step 3) is carried out at a temperature between 20° C. to 70° C. and at a pressure at 0.9 to 6 bar. In one embodiment, the base treatment in step 3) is carried out at a temperature between 15° C. to 35° C. In one embodiment, the base treatment in step 3) is carried out at a temperature between 20° C. to 30° C. In one embodiment, the base treatment in step 3) is carried out at a temperature between 15° C. to 35° C. and at a pressure at approximately 1 bar.

In step 4), the compound according to formula (VIIa) or formula (VIIb) obtained in step 3) is subjected to an acid induced cleavage from the resin, whereby a compound according to formula (VIIIa) or (VIIIb)

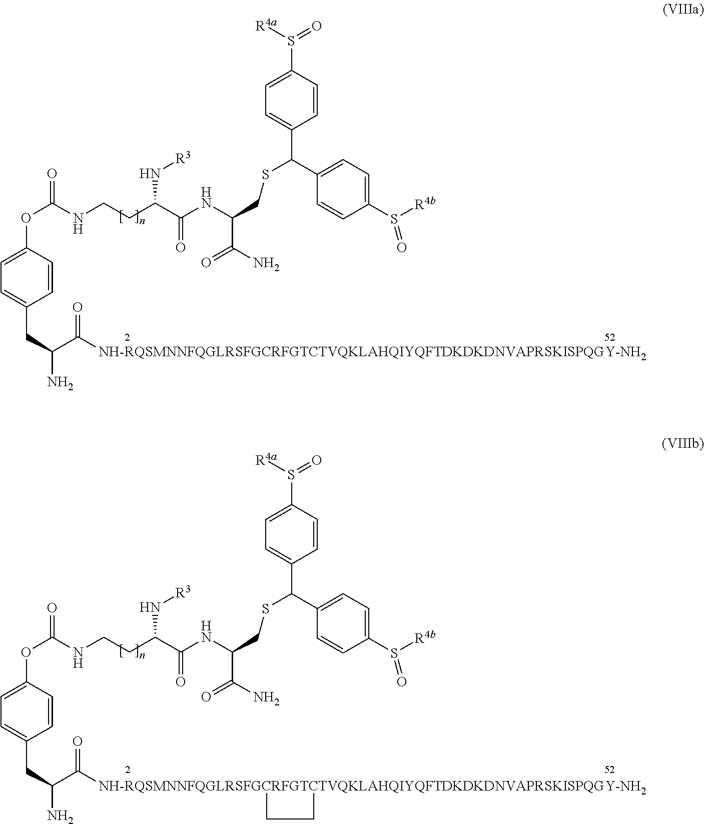

is obtained, wherein
if the compound according to formula (VIIa) or formula (VIIb) comprises as R⁵ a (9H-fluoren-9-ylmethoxy)carbonyl group, said compound is subjected to abase-induced cleavage of the (9H-fluoren-9-ylmethoxy)carbonyl group and then to the acid induced cleavage from the resin, whereby a compound according to formula (VIIIa) or (VIIIb) is obtained;
in which
n represents the number 0, 1 or 2,
R⁵ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R^{4a} represents methyl, ethyl, n-propyl, isopropyl or butyl,
R^{4b} represents methyl, ethyl, n-propyl, isopropyl or butyl.

In one embodiment of step 4), the compound according to formula (VIIa) or formula (VIIb) obtained in step 3) is subjected to an acid induced cleavage from the resin, whereby a compound according to formula (VIIIa) or (VIIIb) is obtained, wherein
if the compound according to formula (VIIa) or formula (VIIb) comprises as R¹ a (9H-fluoren-9-ylmethoxy)carbonyl group, said compound is subjected to a base-induced cleavage of the (9H-fluoren-9-ylmethoxy)carbonyl group and then to the acid induced cleavage from the resin, whereby a compound according to formula (VIIIa) or (VIIIb) is obtained;
in which
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} and R^{4b} represent methyl or ethyl.

In one embodiment of step 4), the compound according to formula (VIIa) or formula (VIIb) obtained in step 3) is subjected to an acid induced cleavage from the resin, whereby a compound according to formula (VIIIa) or (VIIIb) is obtained, wherein
if the compound according to formula (VIIa) or formula (VIIb) comprises as R¹ a (9H-fluoren-9-ylmethoxy)carbonyl group, said compound is subjected to a base-induced cleavage of the (9H-fluoren-9-ylmethoxy)carbonyl group and then to the acid induced cleavage from the resin, whereby a compound according to formula (VIIIa) or (VIIIb) is obtained;
in which
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} represents methyl,
R^{4b} represents methyl.

The compounds according to any one of formula (VIIIa) and/or formula (VIIIb) can be defined as disclosed according to any one of the embodiments described herein.

In step 4), the acid treatment can also induce cleavage of the remaining side chain protecting groups (pg).

In one embodiment, the compound according to formula (VIIa) or formula (VIIb) obtained in step 4) is subjected to an acid induced cleavage from the resin and cleavage of the remaining side chain protecting groups, whereby a compound according to formula (VIIIa) or (VIIIb).

In one embodiment, the acid for the acid induced cleavage in step 4) is selected from trifluoroacetic acid, hydrogen chloride, hydrogen chloride in dioxane and mixtures thereof.

In one embodiment, step 4) is conducted using a cleavage composition comprising at least 80% TFA (V/V) and at least one scavenger selected from water, thiols, silanes and mixtures thereof.

In one embodiment, step 4) is conducted at a temperature between −10° C. and 40° C. In one embodiment, step 4) is conducted at a temperature between −10° C. and 30° C. In one embodiment, step 4) is conducted at a temperature between −10° C. and 40° C. for 1 to 5 hours. In one embodiment, step 4) is conducted at a temperature between −10° C. and 30° C. for 1 to 5 hours.

In one embodiment of step 4), if the compound according to formula (VIIa) or formula (VIIb) comprises as R¹ a (9H-fluoren-9-ylmethoxy)carbonyl group, said compound is subjected to a base-induced cleavage of the (9H-fluoren-9-ylmethoxy)carbonyl group and then to the acid induced cleavage from the resin, whereby a compound according to formula (VIIIa) or (VIIIb) is obtained.

In one embodiment of step 4), the compound according to formula (VIIa) or formula (VIIb) obtained in step 3) is subjected to an acid induced cleavage from the resin, whereby a compound according to formula (VIIIa) or (VIIIb) is obtained, wherein
the compound according to formula (VIIa) or formula (VIIb) is subjected to a base-induced cleavage of the (9H-fluoren-9-ylmethoxy)carbonyl group and
then to the acid induced cleavage from the resin, whereby a compound according to formula (VIIIa) or (VIIIb) is obtained;
in which
n represents the number 1,
R¹ represents (9H-fluoren-9-ylmethoxy)carbonyl,
R³ represents hydrogen, methyl, ethyl, n-propyl or isopropyl,
R⁴ represents methyl, ethyl, n-propyl, isopropyl or butyl,
R^{4b} represents methyl, ethyl, n-propyl, isopropyl or butyl.

In one embodiment of step 4), the compound according to formula (VIIa) or formula (VIIb) obtained in step 3) is subjected to an acid induced cleavage from the resin, whereby a compound according to formula (VIIIa) or (VIIIb) is obtained, wherein
the compound according to formula (VIIa) or formula (VIIb) is subjected to a base-induced cleavage of the (9H-fluoren-9-ylmethoxy)carbonyl group and
then to the acid induced cleavage from the resin, whereby a compound according to formula (VIIIa) or (VIIIb) is obtained;
in which
n represents the number 1,
R¹ represents 9H-fluoren-9-ylmethoxy)carbonyl,
R² represents tert-butyloxycarbonyl,
R³ represents hydrogen or methyl,
R^{4a} and R^{4b} represent methyl or ethyl.

In one embodiment of step 4), the compound according to formula (VIIa) or formula (VIIb) obtained in step 3) is subjected to an acid induced cleavage from the resin, whereby a compound according to formula (VIIIa) or (VIIIb) is obtained, wherein
the compound according to formula (VIIa) or formula (VIIb) is subjected to a base-induced cleavage of the (9H-fluoren-9-ylmethoxy)carbonyl group and
then to the acid induced cleavage from the resin, whereby a compound according to formula (VIIIa) or (VIIIb) is obtained;
in which
n represents the number 1,
R¹ represents 9H-fluoren-9-ylmethoxy)carbonyl,
R² represents tert-butyloxycarbonyl,
R³ represents hydrogen,
R⁴ represents methyl,
R^{4b} represents methyl.

In one embodiment, the base for the base-induced cleavage of the (9H-fluoren-9-ylmethoxy)carbonyl group in step 4) is selected from alkalimetal carbonates, organic bases and mixtures thereof. In one embodiment, the base for the base-induced cleavage of the (9H-fluoren-9-ylmethoxy)carbonyl group in step 4) is selected from alkylamines, dialkylamines and mixtures thereof. In one embodiment, the base for the base-induced cleavage of the (9H-fluoren-9-ylmethoxy)carbonyl group in step 3) is selected from piperidine, 4-methylpiperidine, morpholine, piperazine, pyrrolidine and mixtures thereof.
Step 5) is an optional step. If the product of step 4) is a compound according to formula (VIIIa), said compound is subjected to reaction with an oxidizing agent, whereby a compound according to formula (VIIIb) is obtained,
in which
n represents the number 0, 1 or 2,
R³ represents hydrogen, methyl, ethyl, n-propyl or isopropyl,
R⁴ represents methyl, ethyl, n-propyl, isopropyl or butyl,
R^{4b} represents methyl, ethyl, n-propyl, isopropyl or butyl.

In one embodiment of step 5), the compound according to formula (VIIIa) is subjected to reaction with an oxidizing agent, whereby a compound according to formula (VIIIb) is obtained, in which
n represents the number 1,
R³ represents hydrogen or methyl,
R⁴ represents methyl or ethyl,
R^{4b} represents methyl or ethyl.

In one embodiment of step 5), the compound according to formula (VIIIa) is subjected to reaction with an oxidizing agent, whereby a compound according to formula (VIIIb) is obtained, in which
n represents the number 1,
R³ represents hydrogen,
R⁴ represents methyl,
R^{4b} represents methyl.

The compounds according to any one of formula (VIIIa) and/or formula (VIIIb) can be defined as disclosed according to any one of the embodiments described herein.

In one embodiment, the oxidizing agent in step 5) is selected from iodine, iodine salts, oxygen, H₂O₂, dipyridyl disulfide (DPDS) and mixtures thereof. In one embodiment, the oxidizing agent in step 5) is selected from tetrabutylammonium iodine, NaI, KI, NH₄I and mixtures thereof. In one embodiment, the oxidizing agent in step 5) is NH₄I.

In step 6), the compound according to formula (VIIIb) obtained in step 4) or step 5) is reacted with a cleavage cocktail, whereby a compound according to formula (II)

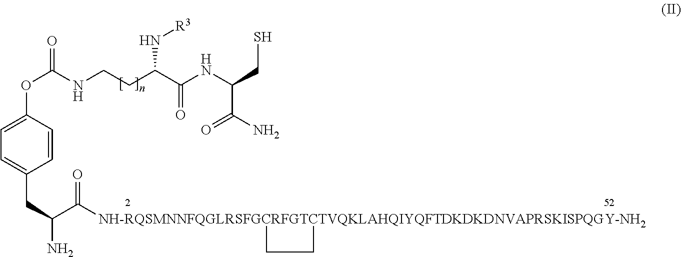

is obtained, in which
n represents the number 0, 1 or 2,
R³ represents hydrogen, methyl, ethyl, n-propyl or isopropyl.

In one embodiment of step 6), the compound according to formula (VIIIb) obtained in step 4) or step 5) is reacted with a cleavage cocktail, whereby a compound according to formula (II) is obtained, in which
n represents the number 1,
R³ represents hydrogen or methyl.

In one embodiment of step 6), the compound according to formula (VIIIb) obtained in step 4) or step 5) is reacted with a cleavage cocktail, whereby a compound according to formula (II) is obtained,
in which
n represents the number 1
R³ represents hydrogen.

The compound according to formula (VIIIb) can be defined as disclosed according to any one of the embodiments described herein.

In one embodiment, the cleavage cocktail in step 6) comprises trifluoroacetic acid, ammonium iodides, and/or mixtures thereof. In one embodiment, the cleavage cocktail in step 6) comprises trifluoroacetic acid, tetra-n-butylammonium iodide (TBAI), ammonium iodide (NH₄I), triisopropylsilane, triethylsilane, and/or mixtures thereof. In one embodiment, the cleavage cocktail in step 6) comprises trifluoroacetic acid. In one embodiment, the cleavage cocktail in step 6) comprises tetra-n-butylammonium iodide (TBAI), ammonium iodide (NH₄I), triisopropylsilane, triethylsilane, and/or mixtures thereof.

In one embodiment, step 6) further comprises separating the compound according to formula (II) thus obtained from the cleavage cocktail. Separating methods are known in the art.

In one embodiment, the separation in step 6) is carried out by precipitation and/or phase separation. In one embodiment, the separation in step 6) is carried out by precipitation using a solvent like tert-butyl methyl ether (MTBE), cyclopentyl methyl ether (CPME), or in particular diisopropyl ether (DIPE).

In step 7), the compound according to formula (II) obtained in step 6) is reacted with a compound according to the formula (IX)

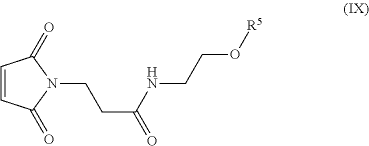

whereby the compound according to formula (I) is obtained, in which R⁵ represents a linear or branched PEG 20 kDa to 80 kDa endcapped with a methoxy-group.

In one embodiment of step 7), R⁵ represents a linear or branched PEG side chain selected from 20 kDa, 25 kDa, 30 kDa, 35 kDa, 40 kDa, 45 kDa, 50 kDa, 55 kDa, 60 kDa, 65 kDa, 70 kDa, 75 kDa and 80 kDa, wherein the PEG is endcapped with a methoxy-group. In one embodiment of step 7), R⁵ represents a linear PEG side chain selected from 20 kDa, 25 kDa, 30 kDa, 35 kDa, 40 kDa, 45 kDa, 50 kDa, 55 kDa, 60 kDa, 65 kDa, 70 kDa, 75 kDa and 80 kDa, wherein the PEG is endcapped with a methoxy-group. In one embodiment of step 7), R⁵ represents a branched PEG side chain selected from 20 kDa, 25 kDa, 30 kDa, 35 kDa, 40 kDa, 45 kDa, 50 kDa, 55 kDa, 60 kDa, 65 kDa, 70 kDa, 75 kDa and 80 kDa, wherein the PEG is endcapped with a methoxy-group.

In one embodiment of step 7), R⁵ represents a linear or branched PEG PEG 30 kDa to 60 kDa endcapped with a methoxy-group. In one embodiment of step 7), R⁵ represents a linear PEG PEG 30 kDa to 60 kDa endcapped with a methoxy-group. In one embodiment of step 7), R⁵ represents a branched PEG PEG 30 kDa to 60 kDa endcapped with a methoxy-group.

In one embodiment of step 7), R⁵ represents a linear or branched PEG 40 kDa endcapped with a methoxy-group. In one embodiment of step 7), R⁵ represents a linear PEG 40 kDa endcapped with a methoxy-group In one embodiment of step 7), R⁵ represents a branched PEG 40 kDa endcapped with a methoxy-group.

In one embodiment, step 7) is conducted in a solvent. In one embodiment, the solvent in step 7) is a buffer or a buffer mixture. In one embodiment, the solvent in step 7) is a buffer or a buffer mixture having a pH 3 to 5. In one embodiment, the solvent in step 7) is a buffer or a buffer mixture having a of pH 4. In one embodiment, the solvent in step 7) is selected from citrate buffers, glycine-hydrochloride buffers, phthalate buffers, acetate buffers and mixtures thereof.

In one embodiment, step 7) is conducted at a temperature range of 0° C. to 50° C. In one embodiment, step 7) is conducted at a temperature range of 5° C. to 30° C. In one embodiment, step 7) is conducted at a temperature range of 0° C. to 50° C. In one embodiment, step 7) is conducted at a temperature range of 10° C. to 25° C.

In the process according to the invention, the compounds according to any one of formulae (I), (II), (III), (IV), (V), (VIIa), (VIIb), (VIIIa), (VIIIb) and/or (X) are used.

In the following, further embodiments of the respective compounds according to any one of formulae (II), (III), (IV), (V), (VIIa), (VIIb), (VIIa), (VIIIb) and/or (X) that can be used in the process according to the invention are disclosed:

In one embodiment, the compound according to any one of formulae (I), (II), (II), (IV), (V), (VIIa), (VIIb), (VIIIa), (VIIIb) and/or (X) is defined as follows
n represents the number 1,
R¹ represents an amine protecting group,
R² represents an amine protecting group,
R³ represents hydrogen, methyl, ethyl, n-propyl or isopropyl,
R^{4a} represents methyl, ethyl, propyl, isopropyl or butyl,
R^{4b} represents methyl, ethyl, propyl, isopropyl or butyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

The amine protecting group can be an acid labile amine protecting group or acid resistant amine protecting group. Amine protecting groups are known in the art.

The amine protecting group can be selected from Carbobenzyloxy (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Acetyl (Ac), Benzoyl (Bz), Benzyl (Bn), Carbamate group, p-Methoxybenzyl (PMB), 3,4-Dimethoxybenzyl (DMPM), p-Methoxyphenyl (PMP), Tosyl (Ts), Troc (trichloroethyl chloroformate) and sulfonamides (Nosyl, Nps). The amine protecting group can be selected from tert-butyloxycarbonyl, (9H-fluoren-9-ylmethoxy)carbonyl and allyloxycarbonyl. The amine protecting group can be tert-butyloxycarbonyl or (9H-fluoren-9-ylmethoxy)carbonyl. The amine protecting group can be allyloxycarbonyl. The amine protecting group can be tert-butyloxycarbonyl. The amine protecting group can be (9H-fluoren-9-ylmethoxy)carbonyl.

The amine protecting group can be an acid labile amine protecting group or acid resistant amine protecting group. Amine protecting groups are known in the art.

The amine protecting group can be selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethylchloroformate) and sulfonamides (Nosyl, Nps).

The amine protecting group can be selected from tert-butyloxycarbonyl, (9H-fluoren-9-ylmethoxy)carbonyl and allyloxycarbonyl. The amine protecting group can be tert-butyloxycarbonyl or (9H-fluoren-9-ylmethoxy)carbonyl. The amine protecting group can be allyloxycarbonyl. The amine protecting group can be tert-butyloxycarbonyl. The amine protecting group can be (9H-fluoren-9-ylmethoxy)carbonyl.

In one embodiment, the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl(FMOC), allyloxycarbonyl(Alloc), Carbamate group, p-Methoxybenzyl(PMB),3,4-Dimethoxybenzyl (DMPM), p-Methoxyphenyl (PMP), Tosyl (Ts), Troc (trichloroethyl chloroformate) and sulfonamides (Nosyl, Nps). In one embodiment, the amine protecting group is selected from tert-butyloxycarbonyl, (9H-fluoren-9-ylmethoxy)carbonyl and allyloxycarbonyl. In one embodiment, the amine protecting group is tert-butyloxycarbonyl or (9H-fluoren-9-ylmethoxy)carbonyl. In one embodiment, the amine protecting group is allyloxycarbonyl. In one embodiment, the amine protecting group is tert-butyloxycarbonyl. In one embodiment, the amine protecting group is (9H-fluoren-9-ylmethoxy)carbonyl.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents an amine protecting group,
R² represents an amine protecting group,
R³ represents hydrogen, methyl, ethyl, n-propyl or isopropyl,
R^{4a} represents methyl, ethyl, propyl, isopropyl or butyl,
R^{4b} represents methyl, ethyl, propyl, isopropyl or butyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof, wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl(FMOC), allyloxycarbonyl(Alloc), Carbamate group, p-Methoxybenzyl(PMB),3,4-Dimethoxybenzyl (DMPM), p-Methoxyphenyl (PMP), Tosyl (Ts), Troc (trichloroethyl chloroformate) and sulfonamides (Nosyl, Nps).

In one embodiment, the compound according to any one of formulae (I), (II), (III), (IV), (V), (VIIa), (VIIb), (VIIIa), (VIIIb) and/or (X) is defined as follows
n represents the number 1,
R¹ represents an amine protecting group,
R² represents an amine protecting group,
R³ represents hydrogen, methyl or ethyl,
R^{4a} represents methyl or ethyl,
R^{4b} represents methyl or ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents an amine protecting group,
R² represents an amine protecting group,
R³ represents hydrogen, methyl or ethyl,
R^{4a} represents methyl or ethyl,
R^{4b} represents methyl or ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof,
wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethylchloroformate) and sulfonamides (Nosyl, Nps).

In one embodiment, the compound according to any one of formulae (I), (II), (III), (IV), (V), (VIIa), (VIIb), (VIIIa), (VIIIb) and/or (X) is defined as follows
n represents the number 1,
R¹ represents an amine protecting group,
R² represents an amine protecting group,
R³ represents hydrogen, methyl or ethyl,
R^{4a} represents methyl or ethyl,
R^{4b} represents methyl or ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof,
wherein the amine protecting group is selected from tert-butyloxycarbonyl, (9H-fluoren-9-ylmethoxy)carbonyl and allyloxycarbonyl.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl, (9H-fluoren-9-ylmethoxy)carbonyl or allyloxycarbonyl,
R² represents tert-butyloxycarbonyl or (9H-fluoren-9-ylmethoxy)carbonyl,
R³ represents hydrogen, methyl or ethyl,
R^{4a} represents methyl or ethyl,
R^{4b} represents methyl or ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl, (9H-fluoren-9-ylmethoxy)carbonyl or allyloxycarbonyl,
R³ represents hydrogen, methyl or ethyl,
R^{4a} represents methyl or ethyl,
R^{4b} represents methyl or ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl, (9H-fluoren-9-ylmethoxy)carbonyl or allyloxycarbonyl,
R² represents tert-butyloxycarbonyl, (9H-fluoren-9-ylmethoxy)carbonyl or allyloxycarbonyl,
R³ represents hydrogen, methyl or ethyl,
R^{4a} represents methyl or ethyl,
R^{4b} represents methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen, methyl or ethyl,
R^{4a} represents methyl or ethyl,
R^{4b} represents methyl or ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIA), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} represents methyl or ethyl,
R^{4b} represents methyl or ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} represents methyl or ethyl,
R^{4b} represents methyl or ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} and R^{4b} represent methyl or ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} and R^{4b} represent methyl or ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIA), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} and R^{4b} represent methyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents an amine protecting group,
R² represents an amine protecting group,
R³ represents hydrogen, methyl, ethyl, n-propyl or isopropyl,
R^{4a} represents methyl, ethyl, propyl, isopropyl or butyl,
R^{4b} represents methyl, ethyl, propyl, isopropyl or butyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents an amine protecting group,
R² represents an amine protecting group,
R³ represents hydrogen, methyl, ethyl, n-propyl or isopropyl,
R^{4a} represents methyl, ethyl, propyl, isopropyl or butyl,
R^{4b} represents methyl, ethyl, propyl, isopropyl or butyl,
R⁵ represents a linear or branched PEG 40 kDa endcapped with a methoxy-group
a hydrate thereof, solvate thereof, salt thereof, pharmaceutically acceptable salt thereof, or the solvates of salts thereof, wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl(FMOC), allyloxycarbonyl(Alloc), Carbamate group, p-Methoxybenzyl(PMB),3,4-Dimethoxybenzyl (DMPM), p-Methoxyphenyl (PMP), Tosyl (Ts), Troc (trichloroethyl chloroformate) and sulfonamides (Nosyl, Nps).

In one embodiment, the compound according to any one of formulae (I), (II), (III), (IV), (V), (VIIa), (VIIb), (VIIIa), (VIIIb) and/or (X) is defined as follows
n represents the number 1,
R¹ represents an amine protecting group,
R² represents an amine protecting group,
R³ represents hydrogen, methyl or ethyl,
R^{4a} represents methyl or ethyl,
R^{4b} represents methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents an amine protecting group,
R² represents an amine protecting group,
R³ represents hydrogen, methyl or ethyl,
R^{4a} represents methyl or ethyl,
R^{4b} represents methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof,
wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethyl chloroformate) and sulfonamides (Nosyl, Nps).

In one embodiment, the compound according to any one of formulae (I), (II), (III), (IV), (V), (VIIa), (VIIb), (VIIIa), (VIIIb) and/or (X) is defined as follows
n represents the number 1,
R¹ represents an amine protecting group,
R² represents an amine protecting group,
R³ represents hydrogen, methyl or ethyl,
R^{4a} represents methyl or ethyl,
R^{4b} represents methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof,
wherein the amine protecting group is selected from tert-butyloxycarbonyl, (9H-fluoren-9-ylmethoxy)carbonyl and allyloxycarbonyl.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl, (9H-fluoren-9-ylmethoxy)carbonyl or allyloxycarbonyl,
R² represents tert-butyloxycarbonyl or (9H-fluoren-9-ylmethoxy)carbonyl,
R³ represents hydrogen, methyl or ethyl,
R^{4a} represents methyl or ethyl,
R^{4b} represents methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl, (9H-fluoren-9-ylmethoxy)carbonyl or allyloxycarbonyl,
R³ represents hydrogen, methyl or ethyl,
R^{4a} represents methyl or ethyl,
R^{4b} represents methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl, (9H-fluoren-9-ylmethoxy)carbonyl or allyloxycarbonyl,
R² represents tert-butyloxycarbonyl, (9H-fluoren-9-ylmethoxy)carbonyl or allyloxycarbonyl,
R³ represents hydrogen, methyl or ethyl,
R^{4a} represents methyl or ethyl,
R^{4b} represents methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen, methyl or ethyl,
R^{4a} represents methyl or ethyl,
R^{4b} represents methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} represents methyl or ethyl,
R^{4b} represents methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} represents methyl or ethyl,
R^{4b} represents methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} and R^{4b} represent methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} and R^{4b} represent methyl or ethyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} and R^{4b} represent methyl,
R⁵ represents a linear or branched PEG 40 kDa endcapped with a methoxy-group
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIa), (VIIb), (VIIIa), (VIIIb) and/or (X) is defined as follows: n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} represents methyl or ethyl,
R^{4b} represents methyl or ethyl,
R⁵ represents a linear or branched PEG 40 kDa endcapped with a methoxy-group,
a hydrate thereof, solvate thereof, salt thereof, pharmaceutically acceptable salt thereof, or the solvates of salts thereof.

In one embodiment, the compound according to any one of formulae (I), (II), (II), (IV), (V), (VIIa), (VIIb), (VIIIa), (VIIIb) and/or (X) is defined as follows: n represents the number 1,
R¹ represents tert-butyloxycarbonyl,
R² represents tert-butyloxycarbonyl,
R³ represents hydrogen or methyl,
R^{4a} and R^{4b} represent methyl or ethyl,
R⁵ represents a linear or branched PEG 40 kDa endcapped with a methoxy-group,
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows
n represents the number 1,
R¹ represents tert-butyloxycarbonyl,
R² represents tert-butyloxycarbonyl,
R³ represents hydrogen,
R^{4a} and R^{4b} represent methyl or ethyl,
R⁵ represents a linear or branched PEG 40 kDa endcapped with a methoxy-group,
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIa), (VIIb), (VIIIa), (VIIIb) and/or (X) is defined as follows: n represents the number 1,
R¹ represents tert-butyloxycarbonyl,
R² represents tert-butyloxycarbonyl,
R³ represents hydrogen,
R^{4a} and R^{4b} represent methyl,
R⁵ represents a linear or branched PEG 40 kDa endcapped with a methoxy-group,
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows: n represents the number 1,
R¹ represents (9H-fluoren-9-ylmethoxy)carbonyl,
R² represents tert-butyloxycarbonyl,
R³ represents hydrogen or methyl,
R^{4a} and R^{4b} represent methyl or ethyl,
R⁵ represents a linear or branched PEG 40 kDa endcapped with a methoxy-group,
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows: n represents the number 1,
R¹ represents (9H-fluoren-9-ylmethoxy)carbonyl,
R² represents tert-butyloxycarbonyl,
R³ represents hydrogen,
R^{4a} and R^{4b} represent methyl or ethyl,
R⁵ represents a linear or branched PEG 40 kDa endcapped with a methoxy-group,
a hydrate thereof, solvate thereof, salt thereof, pharmaceutically acceptable salt thereof, or the solvates of salts thereof.

In one embodiment, the compound according to any one of formulae (I), (II), (III), (IV), (V), (VIIa), (VIIb), (VIIIa), (VIIIb) and/or (X) is defined as follows: n represents the number 1,
R¹ represents (9H-fluoren-9-ylmethoxy)carbonyl,
R² represents tert-butyloxycarbonyl,
R³ represents hydrogen,
R^{4a} and R^{4b} represent methyl,
R⁵ represents a linear or branched PEG 40 kDa endcapped with a methoxy-group,
a hydrate thereof, solvate thereof, salt thereof, pharmaceutically acceptable salt thereof, or the solvates of salts thereof.

Another aspect of the invention refers to the intermediates used in the method of preparation of the compound according to formula (I). One aspect of the invention refers to the compound according to formula (IV)

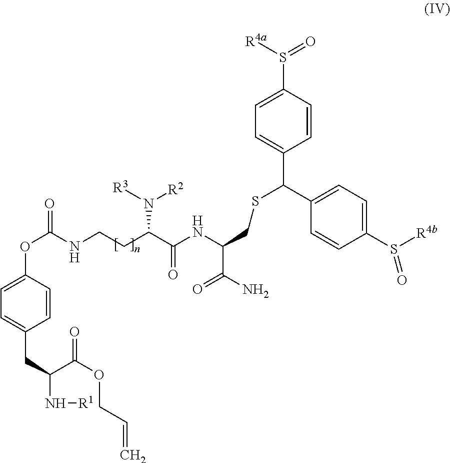

a hydrate thereof, solvate thereof, salt thereof, pharmaceutically acceptable salt thereof, or the solvates of salts thereof.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents an amine protecting group,
R² represents an amine protecting group,
R³ represents hydrogen, methyl, ethyl, n-propyl or isopropyl,
R⁴ represents methyl, ethyl, propyl, isopropyl or butyl,
R^{4b} represents methyl, ethyl, propyl, isopropyl or butyl,
wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethylchloroformate) and sulfonamides (Nosyl, Nps).

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents an amine protecting group,
R² represents an amine protecting group,
R³ represents hydrogen, methyl, ethyl, n-propyl or isopropyl,
R⁴ represents methyl, ethyl, propyl, isopropyl or butyl,
R^{4b} represents methyl, ethyl, propyl, isopropyl or butyl,
wherein the amine protecting group is selected from tert-butyloxycarbonyl, (9H-fluoren-9-ylmethoxy)carbonyl and allyloxycarbonyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents an amine protecting group,
R² represents an amine protecting group,
R³ represents hydrogen, methyl or ethyl,
R^{4a} represents methyl or ethyl,
R^{4b} represents methyl or ethyl,
wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethylchloroformate) and sulfonamides (Nosyl, Nps).

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents tert-butyloxycarbonyl, (9H-fluoren-9-ylmethoxy)carbonyl or allyloxycarbonyl,
R² represents tert-butyloxycarbonyl, (9H-fluoren-9-ylmethoxy)carbonyl or allyloxycarbonyl,
R³ represents hydrogen, methyl or ethyl,
R^{4a} represents methyl or ethyl,
R^{4b} represents methyl or ethyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents tert-butyloxycarbonyl, or (9H-fluoren-9-ylmethoxy)carbonyl,
R² represents tert-butyloxycarbonyl, or (9H-fluoren-9-ylmethoxy)carbonyl,
R³ represents hydrogen, methyl or ethyl,
R^{4a} represents methyl or ethyl,
R^{4b} represents methyl or ethyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R^{4a} represents methyl or ethyl,
R^{4b} represents methyl or ethyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen, methyl or ethyl,
R^{4a} represents methyl or ethyl,
R^{4b} represents methyl or ethyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen, or methyl,
R^{4a} represents methyl or ethyl,
R^{4b} represents methyl or ethyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} represents methyl or ethyl,
R^{4b} represents methyl or ethyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} and R^{4b} represent methyl or ethyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} and R^{4b} represent methyl or ethyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} and R^{4b} represent methyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents tert-butyloxycarbonyl,
R² represents tert-butyloxycarbonyl,
R³ represents hydrogen,
R^{4a} and R^{4b} represent methyl or ethyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents tert-butyloxycarbonyl,
R² represents tert-butyloxycarbonyl,
R³ represents hydrogen,
R^{4a} and R^{4b} represent methyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents (9H-fluoren-9-ylmethoxy)carbonyl,
R² represents tert-butyloxycarbonyl,
R³ represents hydrogen,
R^{4a} and R^{4b} represent methyl or ethyl.

In one embodiment, the compound according to formula (IV), a hydrate thereof, solvate thereof, salt thereof, pharmaceutically acceptable salt thereof, or the solvates of salts thereof is defined as follows:
n represents the number 1,
R¹ represents (9H-fluoren-9-ylmethoxy)carbonyl,
R² represents tert-butyloxycarbonyl,
R³ represents hydrogen,
R^{4a} and R^{4b} represent methyl.

Another aspect of the invention refers to a process for the preparation of the compound according to formula (IV).

In one embodiment the compound according to formula (IV), a hydrate thereof, solvate thereof, salt thereof, pharmaceutically acceptable salt thereof, or the solvates of salts thereof is prepared as follows:
Step 1.1.b): Reacting a compound according to formula (X) with a compound according to formula (III) whereby a compound according to formula (IV) is obtained, in which
n represents the number 0, 1 or 2,
R¹ represents an amine protecting group,
R² represents an amine protecting group,
R³ represents hydrogen, methyl, ethyl, n-propyl or isopropyl,
R⁴ represents methyl, ethyl, n-propyl, isopropyl or butyl,
R^{4b} represents methyl, ethyl, n-propyl, isopropyl or butyl,
wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethyl chloroformate) and sulfonamides (Nosyl, Nps).

In one embodiment the compound according to formula (IV), a hydrate thereof, solvate thereof, salt thereof, pharmaceutically acceptable salt thereof, or the solvates of salts thereof is prepared as follows:
Step 1.1.b): Reacting a compound according to formula (X) with a compound according to formula (III) whereby a compound according to formula (IV) is obtained, in which
n represents the number 0, 1 or 2,
R¹ represents an amine protecting group,
R² represents an amine protecting group,
R³ represents hydrogen, methyl, ethyl, n-propyl or isopropyl,
R⁴ represents methyl, ethyl, n-propyl, isopropyl or butyl,
R^{4b} represents methyl, ethyl, n-propyl, isopropyl or butyl,
wherein the amine protecting group is selected from tert-butyloxycarbonyl, (9H-fluoren-9-ylmethoxy)carbonyl and allyloxycarbonyl.

In one embodiment the compound according to formula (IV), a hydrate thereof, solvate thereof, salt thereof, pharmaceutically acceptable salt thereof, or the solvates of salts thereof is prepared as follows:
Step 1.1.b): Reacting a compound according to formula (X) with a compound according to formula (III) whereby a compound according to formula (IV) is obtained, in which
n represents the number 0, 1 or 2,
R¹ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R^{4a} represents methyl, ethyl, n-propyl, isopropyl or butyl,
R^{4b} represents methyl, ethyl, n-propyl, isopropyl or butyl.

In one embodiment the compound according to formula (IV), a hydrate thereof, solvate thereof, salt thereof, pharmaceutically acceptable salt thereof, or the solvates of salts thereof is prepared as follows:
Step 1.1.b): Reacting a compound according to formula (X) with a compound according to formula (I-1) whereby a compound according to formula (IV) is obtained.

In one embodiment the compound according to formula (IV), a hydrate thereof, solvate thereof, salt thereof, pharmaceutically acceptable salt thereof, or the solvates of salts thereof is prepared as follows:
Step 1.1.b): Reacting a compound according to formula (X) with a compound according to formula (I-1)
whereby a compound according to formula (IV) is obtained, in which
n represents the number 1,
R¹ represents an amine protecting group,
R² represents an amine protecting group,
R³ represents hydrogen, methyl or ethyl,
wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethylchloroformate) and sulfonamides (Nosyl, Nps).

In one embodiment the compound according to formula (IV), a hydrate thereof, solvate thereof, salt thereof, pharmaceutically acceptable salt thereof, or the solvates of salts thereof is prepared as follows:
Step 1.1.b): Reacting a compound according to formula (X) with a compound according to formula (I-1)
whereby a compound according to formula (IV) is obtained, in which
n represents the number 0, 1 or 2,
R¹ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R^{4a} represents methyl,
R^{4b} represents methyl.

In one embodiment the compound according to formula (IV), a hydrate thereof, solvate thereof, salt thereof, pharmaceutically acceptable salt thereof, or the solvates of salts thereof is prepared as follows:
Step 1.1.b): Reacting a compound according to formula (X) with a compound according to formula (I-1)
whereby a compound according to formula (IV) is obtained, in which
n represents the number 0, 1 or 2,
R¹ represents tert-butyloxycarbonyl or (9H-fluoren-9-ylmethoxy)carbonyl,
R² represents tert-butyloxycarbonyl,
R³ represents hydrogen.

In one embodiment the compound according to formula (IV), a hydrate thereof, solvate thereof, salt thereof, pharmaceutically acceptable salt thereof, or the solvates of salts thereof is prepared as follows:
Step 1.1.b): Reacting a compound according to formula (X) with a compound according to formula (I-1)
whereby a compound according to formula (IV) is obtained, in which
n represents the number 1,
R¹ represents tert-butyloxycarbonyl or (9H-fluoren-9-ylmethoxy)carbonyl,
R² represents tert-butyloxycarbonyl,
R³ represents hydrogen.

Further embodiments of step 1.1.b) are described above. These embodiments can be used for the preparation of the compound according to formula (IV). The embodiments of step 1.1.b) described above can be used for the preparation of the compound according to formula (IV) disclosed above.

A further aspect of the invention is the use of the compound according to formula (IV) in the process for the preparation of the compound according to formula (I) as described herein. A further aspect of the invention is the use of the compound according to formula (IV) in the process for the preparation of the compound according to formula (Ia) as described herein. A further aspect of the invention is the use of the compound according to formula (IV) in the process for the preparation of the compound according to formula (VIIa) as described herein. A further aspect of the invention is the use of the compound according to formula (IV) in the process for the preparation of the compound according to formula (VIIb) as described herein. A further aspect of the invention is the use of the compound according to formula (IV) in the process for the preparation of the compound according to formula (VIIIa) as described herein. A further aspect of the invention is the use of the compound according to formula (IV) in the process for the preparation of the compound according to formula (VIIIb) as described herein.

One aspect of the invention refers to the compound according to formula (VIIa)

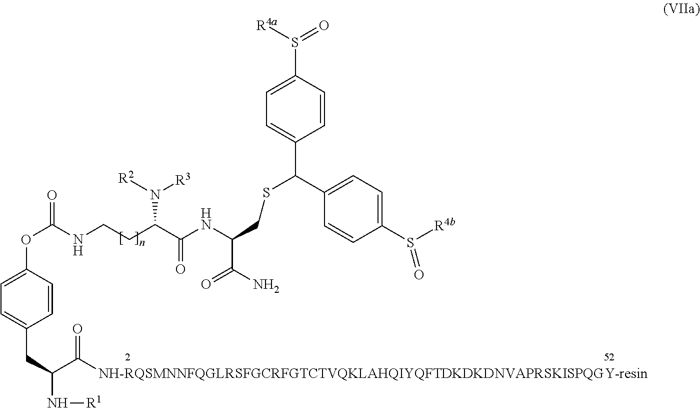

a hydrate thereof, solvate thereof, salt thereof, pharmaceutically acceptable salt thereof, or the solvates of salts thereof.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents an amine protecting group,
R² represents an amine protecting group,
R³ represents hydrogen, methyl, ethyl, n-propyl or isopropyl,
R^{4a} represents methyl, ethyl, propyl, isopropyl or butyl,
R^{4b} represents methyl, ethyl, propyl, isopropyl or butyl,
wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethyl chloroformate) and sulfonamides (Nosyl, Nps

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents an amine protecting group,
R² represents an amine protecting group,
R³ represents hydrogen, methyl, ethyl, n-propyl or isopropyl,
R^{4a} represents methyl, ethyl, propyl, isopropyl or butyl,
R^{4b} represents methyl, ethyl, propyl, isopropyl or butyl,
wherein the amine protecting group is selected from tert-butyloxycarbonyl, (9H-fluoren-9-ylmethoxy)carbonyl and allyloxycarbonyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents an amine protecting group,
R² represents an amine protecting group,
R³ represents hydrogen, methyl or ethyl,
R^{4a} represents methyl or ethyl,
R^{4b} represents methyl or ethyl,
wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethylchloroformate) and sulfonamides (Nosyl, Nps).

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents tert-butyloxycarbonyl, (9H-fluoren-9-ylmethoxy)carbonyl or allyloxycarbonyl,
R² represents tert-butyloxycarbonyl, (9H-fluoren-9-ylmethoxy)carbonyl or allyloxycarbonyl,
R³ represents hydrogen, methyl or ethyl,
R^{4a} represents methyl or ethyl,
R^{4b} represents methyl or ethyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents tert-butyloxycarbonyl, or (9H-fluoren-9-ylmethoxy)carbonyl,
R² represents tert-butyloxycarbonyl, or (9H-fluoren-9-ylmethoxy)carbonyl,
R³ represents hydrogen, methyl or ethyl,
R^{4a} represents methyl or ethyl,
R^{4b} represents methyl or ethyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R^{4a} represents methyl or ethyl,
R^{4b} represents methyl or ethyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen, methyl, or ethyl,
R^{4a} represents methyl or ethyl,
R^{4b} represents methyl or ethyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} represents methyl or ethyl,
R^{4b} represents methyl or ethyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} represents methyl or ethyl,
R^{4b} represents methyl or ethyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} represents methyl or ethyl,
R^{4b} represents methyl or ethyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} and R^{4b} represent methyl or ethyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} and R^{4b} represent methyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents tert-butyloxycarbonyl,
R² represents tert-butyloxycarbonyl,
R³ represents hydrogen,
R^{4a} and R^{4b} represent methyl or ethyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents tert-butyloxycarbonyl,
R² represents tert-butyloxycarbonyl,
R³ represents hydrogen,
R^{4a} and R^{4b} represent methyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents (9H-fluoren-9-ylmethoxy)carbonyl,
R² represents tert-butyloxycarbonyl,
R³ represents hydrogen,
R^{4a} and R^{4b} represent methyl or ethyl.

In one embodiment, the compound according to formula (VIIa), a hydrate thereof, solvate thereof, salt thereof, pharmaceutically acceptable salt thereof, or the solvates of salts thereof is defined as follows:
n represents the number 1,
R¹ represents (9H-fluoren-9-ylmethoxy)carbonyl,
R² represents tert-butyloxycarbonyl,
R³ represents hydrogen,
R^{4a} and R^{4b} represent methyl.

Another aspect of the invention refers to a process for the preparation of the compound according to formula (VIIa).

In one embodiment the compound according to formula (VIIa), a hydrate thereof, solvate thereof, salt thereof, pharmaceutically acceptable salt thereof, or the solvates of salts thereof is prepared as follows:
Step 3) Reacting the compound according to formula (V) with a compound according to formula (VIa), whereby a compound according to formula (VIIa) is obtained.

Embodiments of step 3) are described in detail above. These embodiments can be used for the preparation of the compound according to formula (VIIa).

In one embodiment the compound according to formula (VIIa), a hydrate thereof, solvate thereof, salt thereof, pharmaceutically acceptable salt thereof, or the solvates of salts thereof is prepared as follows:
Step 1) Providing a compound according to formula (V);
Step 2) Providing a compound according to formula (VIa);
Step 3) Reacting the compound according to formula (V) with a compound according to formula (VIa), whereby a compound according to formula (VIIa) is obtained.

Embodiments of step 1), step 2) and/or step 3) are described in detail above. These embodiments can be used for the preparation of the compound according to formula (VIIa).

In one embodiment the compound according to formula (VIIa), a hydrate thereof, solvate thereof, salt thereof, pharmaceutically acceptable salt thereof, or the solvates of salts thereof is prepared as follows:
Step 1.1.a) Providing a compound according to formula (IV);
Step 1.2) Reacting the compound according to formula (IV) with a palladium(0) source and a nucleophile, whereby a compound according to formula (V) is obtained;
Step 2) Providing a compound according to formula (VIa);
Step 3) Reacting the compound according to formula (V) with a compound according to formula (VIa), whereby a compound according to formula (VIIa) is obtained.

Embodiments of step 1.1.a), step 1.2), step 2) and/or step 3) are described in detail above. These embodiments can be used for the preparation of the compound according to formula (Via).

In one embodiment the compound according to formula (VIIa), a hydrate thereof, solvate thereof, salt thereof, pharmaceutically acceptable salt thereof, or the solvates of salts thereof is prepared as follows:
Step 1.1.b) Reacting a compound according to formula (X) with a compound according to formula (III) whereby a compound according to formula (IV) is obtained;
Step 1.2) Reacting the compound according to formula (IV) with a palladium(0) source and a nucleophile, whereby a compound according to formula (V) is obtained;
Step 2) Providing a compound according to formula (VIa);
Step 3) Reacting the compound according to formula (V) with a compound according to formula (VIa), whereby a compound according to formula (VIIa) is obtained.

Embodiments of step 1.1.b), step 1.2), step 2) and/or step 3) are described in detail above. These embodiments can be used for the preparation of the compound according to formula (Via).

A further aspect of the invention is the use of the compound according to formula (VIIa) in the process for the preparation of the compound according to formula (I). A further aspect of the invention is the use of the compound according to formula (VIIa) in the process for the preparation of the compound according to formula (Ia). A further aspect of the invention is the use of the compound according to formula (VIIa) in the process for the preparation of the compound according to formula (VIIIa) as described herein. A further aspect of the invention is the use of the compound according to formula (VIIa) in the process for the preparation of the compound according to formula (VIIIb) as described herein.

One aspect of the invention refers to the compound according to formula (VIIb)

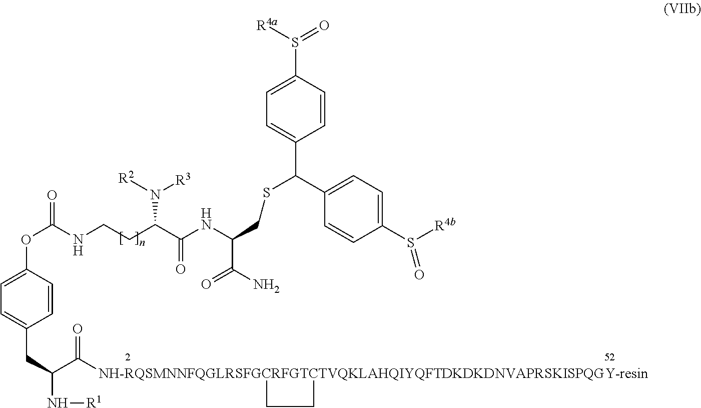

a hydrate thereof, solvate thereof, salt thereof, pharmaceutically acceptable salt thereof, or the solvates of salts thereof.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents an amine protecting group,
R² represents an amine protecting group,
R³ represents hydrogen, methyl, ethyl, n-propyl or isopropyl,
R^{4a} represents methyl, ethyl, propyl, isopropyl or butyl,
R^{4b} represents methyl, ethyl, propyl, isopropyl or butyl,
wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethyl chloroformate) and sulfonamides (Nosyl, Nps

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents an amine protecting group,
R² represents an amine protecting group,
R³ represents hydrogen, methyl, ethyl, n-propyl or isopropyl,
R^{4a} represents methyl, ethyl, propyl, isopropyl or butyl,
R^{4b} represents methyl, ethyl, propyl, isopropyl or butyl,
wherein the amine protecting group is selected from tert-butyloxycarbonyl, (9H-fluoren-9-ylmethoxy)carbonyl and allyloxycarbonyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents an amine protecting group,
R² represents an amine protecting group,
R³ represents hydrogen, methyl or ethyl,
R^{4a} represents methyl or ethyl,
R^{4b} represents methyl or ethyl,
wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethylchloroformate) and sulfonamides (Nosyl, Nps).

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents tert-butyloxycarbonyl, (9H-fluoren-9-ylmethoxy)carbonyl or allyloxycarbonyl,
R² represents tert-butyloxycarbonyl, (9H-fluoren-9-ylmethoxy)carbonyl or allyloxycarbonyl,
R³ represents hydrogen, methyl or ethyl,
R^{4a} represents methyl or ethyl,
R^{4b} represents methyl or ethyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents tert-butyloxycarbonyl, or (9H-fluoren-9-ylmethoxy)carbonyl,
R² represents tert-butyloxycarbonyl, or (9H-fluoren-9-ylmethoxy)carbonyl,
R³ represents hydrogen, methyl or ethyl,
R^{4a} represents methyl or ethyl,
R^{4b} represents methyl or ethyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R^{4a} represents methyl or ethyl,
R^{4b} represents methyl or ethyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen, methyl or ethyl,
R^{4a} represents methyl or ethyl,
R^{4b} represents methyl or ethyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} represents methyl or ethyl,
R^{4b} represents methyl or ethyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} represents methyl or ethyl,
R^{4b} represents methyl or ethyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen or methyl,
R^{4a} and R^{4b} represent methyl or ethyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} and R^{4b} represent methyl or ethyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents tert-butyloxycarbonyl or (9H-fluoren-9-ylmethoxy)carbonyl,
R² represents tert-butyloxycarbonyl,
R³ represents hydrogen,
R^{4a} and R^{4b} represent methyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents tert-butyloxycarbonyl,
R² represents tert-butyloxycarbonyl,
R³ represents hydrogen,
R^{4a} and R^{4b} represent methyl or ethyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents tert-butyloxycarbonyl,
R² represents tert-butyloxycarbonyl,
R³ represents hydrogen,
R^{4a} and R^{4b} represent methyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents (9H-fluoren-9-ylmethoxy)carbonyl,
R² represents tert-butyloxycarbonyl,
R³ represents hydrogen,
R^{4a} and R^{4b} represent methyl or ethyl.

In one embodiment, the compound according to formula (VIIb), a hydrate thereof, solvate thereof, salt thereof, pharmaceutically acceptable salt thereof, or the solvates of salts thereof is defined as follows:
n represents the number 1,
R¹ represents (9H-fluoren-9-ylmethoxy)carbonyl,
R² represents tert-butyloxycarbonyl,
R³ represents hydrogen,
R^{4a} and R^{4b} represent methyl.

Another aspect of the invention refers to a process for the preparation of the compound according to formula (VIIb).

In one embodiment the compound according to formula (VIIb), a hydrate thereof, solvate thereof, salt thereof, pharmaceutically acceptable salt thereof, or the solvates of salts thereof is prepared as follows:
Step 3) Reacting the compound according to formula (V) with a compound according to formula (VIb), whereby a compound according to formula (VIIb) is obtained.

Embodiments of step 3) are described in detail above. These embodiments can be used for the preparation of the compound according to formula (VIIb).

In one embodiment the compound according to formula (VIIb), a hydrate thereof, solvate thereof, salt thereof, pharmaceutically acceptable salt thereof, or the solvates of salts thereof is prepared as follows:
Step 1) Providing a compound according to formula (V);
Step 2) Providing a compound according to formula (VIb);
Step 3) Reacting the compound according to formula (V) with a compound according to formula (VIb), whereby a compound according to formula (VIIb) is obtained.

Embodiments of step 1), step 2) and/or step 3) are described in detail above. These embodiments can be used for the preparation of the compound according to formula (VIIb).

In one embodiment the compound according to formula (VIIb), a hydrate thereof, solvate thereof, salt thereof, pharmaceutically acceptable salt thereof, or the solvates of salts thereof is prepared as follows:
Step 1.1.a) Providing a compound according to formula (IV);
Step 1.2) Reacting the compound according to formula (IV) with a palladium(0) source and a nucleophile, whereby a compound according to formula (V) is obtained;
Step 2) Providing a compound according to formula (VIb);
Step 3) Reacting the compound according to formula (V) with a compound according to formula (VIb), whereby a compound according to formula (VIIb) is obtained.

Embodiments of step 1.1.a), step 1.2), step 2) and/or step 3) are described in detail above. These embodiments can be used for the preparation of the compound according to formula (VIIb).

In one embodiment the compound according to formula (VIIb), a hydrate thereof, solvate thereof, salt thereof, pharmaceutically acceptable salt thereof, or the solvates of salts thereof is prepared as follows:
Step 1.1.b) Reacting a compound according to formula (X) with a compound according to formula (III) whereby a compound according to formula (IV) is obtained;
Step 1.2) Reacting the compound according to formula (IV) with a palladium(0) source and a nucleophile, whereby a compound according to formula (V) is obtained;
Step 2) Providing a compound according to formula (VIb);
Step 3) Reacting the compound according to formula (V) with a compound according to formula (VIb), whereby a compound according to formula (VIIb) is obtained.

Embodiments of step 1.1.b), step 1.2), step 2) and/or step 3) are described in detail above. These embodiments can be used for the preparation of the compound according to formula (VIIb).

A further aspect of the invention is the use of the compound according to formula (VIIb) in the process for the preparation of the compound according to formula (I). A further aspect of the invention is the use of the compound according to formula (VIIb) in the process for the preparation of the compound according to formula (Ia). A further aspect of the invention is the use of the compound according to formula (VIIb) in the process for the preparation of the compound according to formula (Ia). A further aspect of the invention is the use of the compound according to formula (VIIb) in the process for the preparation of the compound according to formula (VIIIb) as described herein.

One aspect of the invention refers to the compound according to formula (VIIIa)

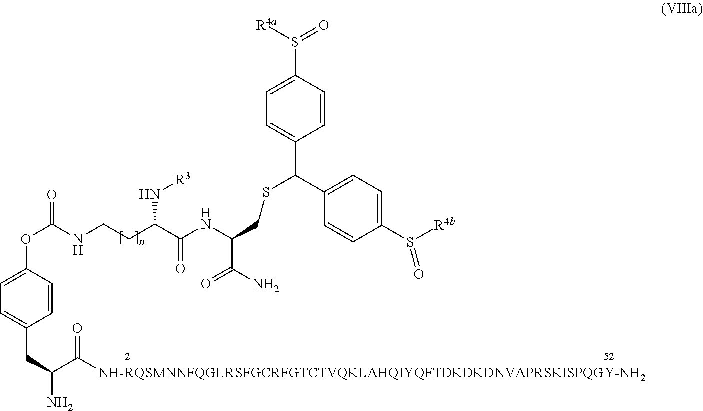

a hydrate thereof, solvate thereof, salt thereof, pharmaceutically acceptable salt thereof, or the solvates of salts thereof.

In one embodiment, the compound according to formula (VIIIa), a hydrate thereof, solvate thereof, salt thereof, pharmaceutically acceptable salt thereof, or the solvates of salts thereof is defined as follows:
n represents the number 1,
R³ represents hydrogen, methyl, ethyl, n-propyl or isopropyl,
R^{4a} represents methyl or ethyl,
R^{4b} represents methyl or ethyl.

In one embodiment, the compound according to formula (VIIIa), a hydrate thereof, solvate thereof, salt thereof, pharmaceutically acceptable salt thereof, or the solvates of salts thereof is defined as follows:
n represents the number 1,
R³ represents hydrogen, methyl or ethyl,
R^{4a} represents methyl or ethyl,
R^{4b} represents methyl or ethyl.

In one embodiment, the compound according to formula (VIIIa), a hydrate thereof, solvate thereof, salt thereof, pharmaceutically acceptable salt thereof, or the solvates of salts thereof is defined as follows:
n represents the number 1,
R³ represents hydrogen or methyl,
R^{4a} represents methyl or ethyl,
R^{4b} represents methyl or ethyl.

In one embodiment, the compound according to formula (VIIIa), a hydrate thereof, solvate thereof, salt thereof, pharmaceutically acceptable salt thereof, or the solvates of salts thereof is defined as follows:
n represents the number 1,
R³ represents hydrogen,
R^{4a} represents methyl or ethyl,
R^{4b} represents methyl or ethyl.

In one embodiment, the compound according to formula (VIIIa), a hydrate thereof, solvate thereof, salt thereof, pharmaceutically acceptable salt thereof, or the solvates of salts thereof is defined as follows:
n represents the number 1,
R³ represents hydrogen or methyl,
R^{4a} and R^{4b} represent methyl or ethyl.

In one embodiment, the compound according to formula (VIIIa), a hydrate thereof, solvate thereof, salt thereof, pharmaceutically acceptable salt thereof, or the solvates of salts thereof is defined as follows:
n represents the number 1,
R³ represents hydrogen,
R^{4a} and R^{4b} represent methyl or ethyl.

In one embodiment, the compound according to formula (VIIIa), a hydrate thereof, solvate thereof, salt thereof, pharmaceutically acceptable salt thereof, or the solvates of salts thereof is defined as follows:
n represents the number 1,
R³ represents hydrogen,
R^{4a} and R^{4b} represent methyl.

Another aspect of the invention refers to a process for the preparation of the compound according to formula (VIIIa).

In one embodiment the compound according to formula (VIIIa), a hydrate thereof, solvate thereof, salt thereof, pharmaceutically acceptable salt thereof, or the solvates of salts thereof is prepared as follows:
Step 4) Subjecting the compound according to formula (VIIa) to an acid induced cleavage from the resin, whereby the compound according to formula (VIIIa) is obtained.

Embodiments of step 4) are described in detail above. These embodiments can be used for the preparation of the compound according to formula (VIIIa).

In one embodiment the compound according to formula (VIIIa), a hydrate thereof, solvate thereof, salt thereof, pharmaceutically acceptable salt thereof, or the solvates of salts thereof is prepared as follows:
Step 3) Reacting the compound according to formula (V) with a compound according to formula (VIa), whereby a compound according to formula (VIIa) is obtained;
Step 4) Subjecting the compound according to formula (VIIa) to an acid induced cleavage from the resin, whereby the compound according to formula (VIIIa) is obtained.

Embodiments of step 3) and/or step 4) are described in detail above. These embodiments can be used for the preparation of the compound according to formula (VIIIa).

In one embodiment the compound according to formula (VIIIa), a hydrate thereof, solvate thereof, salt thereof, pharmaceutically acceptable salt thereof, or the solvates of salts thereof is prepared as follows:
Step 1) Providing a compound according to formula (V);
Step 2) Providing a compound according to formula (VIa);
Step 3) Reacting the compound according to formula (V) with a compound according to formula (VIa), whereby a compound according to formula (VIIa) is obtained;
Step 4) Subjecting the compound according to formula (VIIa) to an acid induced cleavage from the resin, whereby the compound according to formula (VIIIa) is obtained.

Embodiments of step 1), step 2), step 3) and/or step 4) are described in detail above. These embodiments can be used for the preparation of the compound according to formula (VIIIa).

In one embodiment the compound according to formula (VIIIa), a hydrate thereof, solvate thereof, salt thereof, pharmaceutically acceptable salt thereof, or the solvates of salts thereof is prepared as follows:
Step 1.1.a) Providing a compound according to formula (IV);
Step 1.2) Reacting the compound according to formula (IV) with a palladium(0) source and a nucleophile, whereby a compound according to formula (V) is obtained;
Step 2) Providing a compound according to formula (VIa);
Step 3) Reacting the compound according to formula (V) with a compound according to formula (VIa), whereby a compound according to formula (VIIa) is obtained;
Step 4) Subjecting the compound according to formula (VIIa) to an acid induced cleavage from the resin, whereby the compound according to formula (VIIIa) is obtained.

Embodiments of step 1.1.a), step 1.2), step 2), step 3) and/or step 4) are described in detail above. These embodiments can be used for the preparation of the compound according to formula (VIa).

In one embodiment the compound according to formula (VIIIa), a hydrate thereof, solvate thereof, salt thereof, pharmaceutically acceptable salt thereof, or the solvates of salts thereof is prepared as follows:
Step 1.1.b) Reacting a compound according to formula (X) with a compound according to formula (III) whereby a compound according to formula (IV) is obtained;
Step 1.2) Reacting the compound according to formula (IV) with a palladium(0) source and a nucleophile, whereby a compound according to formula (V) is obtained;
Step 2) Providing a compound according to formula (VIa);
Step 3) Reacting the compound according to formula (V) with a compound according to formula (VIa), whereby a compound according to formula (VIIa) is obtained;
Step 4) Subjecting the compound according to formula (VIIa) to an acid induced cleavage from the resin, whereby the compound according to formula (VIIIa) is obtained.

Embodiments of step 1.1.b), step 1.2), step 2), step 3) and/or step 4) are described in detail above. These embodiments can be used for the preparation of the compound according to formula (VIIIa).

A further aspect of the invention is the use of the compound according to formula (VIIIa) in the process for the preparation of the compound according to formula (I). A further aspect of the invention is the use of the compound according to formula (VIIIa) in the process for the preparation of the compound according to formula (Ia). A further aspect of the invention is the use of the compound according to formula (VIIIa) in the process for the preparation of the compound according to formula (VIIIb) as described herein.

One aspect of the invention refers to the compound according to formula (VIIIb).

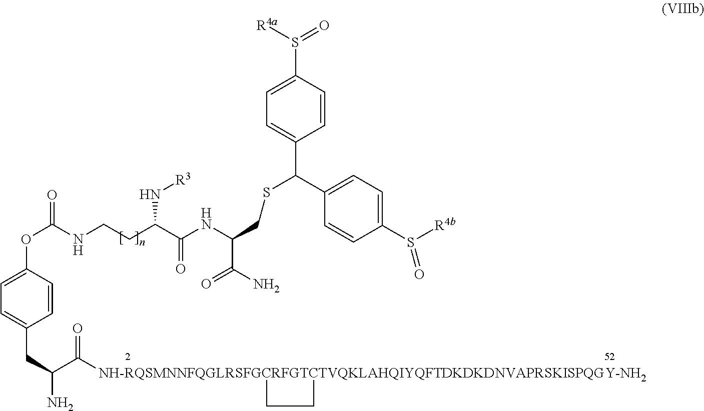

a hydrate thereof, solvate thereof, salt thereof, pharmaceutically acceptable salt thereof, or the solvates of salts thereof.

In one embodiment, the compound according to formula (VIIIb), a hydrate thereof, solvate thereof, salt thereof, pharmaceutically acceptable salt thereof, or the solvates of salts thereof is defined as follows:
n represents the number 1,
R³ represents hydrogen, methyl, ethyl, n-propyl or isopropyl,
R^{4a} represents methyl or ethyl,
R^{4b} represents methyl or ethyl.

In one embodiment, the compound according to formula (VIIIb), a hydrate thereof, solvate thereof, salt thereof, pharmaceutically acceptable salt thereof, or the solvates of salts thereof is defined as follows:
n represents the number 1,
R³ represents hydrogen, methyl or ethyl,
R^{4a} represents methyl or ethyl,
R^{4b} represents methyl or ethyl.

In one embodiment, the compound according to formula (VIIIb), a hydrate thereof, solvate thereof, salt thereof, pharmaceutically acceptable salt thereof, or the solvates of salts thereof is defined as follows:
n represents the number 1,
R³ represents hydrogen or methyl,
R^{4a} represents methyl or ethyl,
R^{4b} represents methyl or ethyl.

In one embodiment, the compound according to formula (VIIIb), a hydrate thereof, solvate thereof, salt thereof, pharmaceutically acceptable salt thereof, or the solvates of salts thereof is defined as follows:
n represents the number 1,
R³ represents hydrogen,
R^{4a} represents methyl or ethyl,
R^{4b} represents methyl or ethyl.

In one embodiment, the compound according to formula (VIIIb), a hydrate thereof, solvate thereof, salt thereof, pharmaceutically acceptable salt thereof, or the solvates of salts thereof is defined as follows:
n represents the number 1,
R³ represents hydrogen or methyl,
R^{4a} and R^{4b} represent methyl or ethyl.

In one embodiment, the compound according to formula (VIIIb), a hydrate thereof, solvate thereof, salt thereof, pharmaceutically acceptable salt thereof, or the solvates of salts thereof is defined as follows:
n represents the number 1,
R³ represents hydrogen,
R^{4a} and R^{4b} represent methyl or ethyl.

In one embodiment, the compound according to formula (VIIIb), a hydrate thereof, solvate thereof, salt thereof, pharmaceutically acceptable salt thereof, or the solvates of salts thereof is defined as follows:
n represents the number 1,
R³ represents hydrogen,
R^{4a} and R^{4b} represent methyl.

Another aspect of the invention refers to a process for the preparation of the compound according to formula (VIIIb).

In one embodiment the compound according to formula (VIIIb), a hydrate thereof, solvate thereof, salt thereof, pharmaceutically acceptable salt thereof, or the solvates of salts thereof is prepared as follows:
Step 4) Subjecting the compound according to formula (VIIb) to an acid induced cleavage from the resin, whereby the compound according to formula (VIIIb) is obtained.

Embodiments of step 4) are described in detail above. These embodiments can be used for the preparation of the compound according to formula (VIIIb).

In one embodiment the compound according to formula (VIIIb), a hydrate thereof, solvate thereof, salt thereof, pharmaceutically acceptable salt thereof, or the solvates of salts thereof is prepared as follows:
Step 3) Reacting the compound according to formula (V) with a compound according to formula (VIb), whereby a compound according to formula (VIIb) is obtained;
Step 4) Subjecting the compound according to formula (VIIb) to an acid induced cleavage from the resin, whereby the compound according to formula (VIIIb) is obtained.

Embodiments of step 3) and/or step 4) are described in detail above. These embodiments can be used for the preparation of the compound according to formula (VIIIb).

In one embodiment the compound according to formula (VIIIb), a hydrate thereof, solvate thereof, salt thereof, pharmaceutically acceptable salt thereof, or the solvates of salts thereof is prepared as follows:
Step 1) Providing a compound according to formula (V);
Step 2) Providing a compound according to formula (VIb);
Step 3) Reacting the compound according to formula (V) with a compound according to formula (VIb), whereby a compound according to formula (VIIb) is obtained;
Step 4) Subjecting the compound according to formula (VIIb) to an acid induced cleavage from the resin, whereby the compound according to formula (VIIIb) is obtained.

Embodiments of step 1), step 2), step 3) and/or step 4) are described in detail above. These embodiments can be used for the preparation of the compound according to formula (VIIIb).

In one embodiment the compound according to formula (VIIIb), a hydrate thereof, solvate thereof, salt thereof, pharmaceutically acceptable salt thereof, or the solvates of salts thereof is prepared as follows:
Step 1.1.a) Providing a compound according to formula (IV);
Step 1.2) Reacting the compound according to formula (IV) with a palladium(0) source and a nucleophile, whereby a compound according to formula (V) is obtained;
Step 2) Providing a compound according to formula (VIb);
Step 3) Reacting the compound according to formula (V) with a compound according to formula (VIb), whereby a compound according to formula (VIIb) is obtained;
Step 4) Subjecting the compound according to formula (VIIb) to an acid induced cleavage from the resin, whereby the compound according to formula (VIIIb) is obtained.

Embodiments of step 1.1.a), step 1.2), step 2), step 3) and/or step 4) are described in detail above. These embodiments can be used for the preparation of the compound according to formula (VIIIb).

In one embodiment the compound according to formula (VIIIb), a hydrate thereof, solvate thereof, salt thereof, pharmaceutically acceptable salt thereof, or the solvates of salts thereof is prepared as follows:
Step 1.1.b) Reacting a compound according to formula (X) with a compound according to formula (III) whereby a compound according to formula (IV) is obtained;
Step 1.2) Reacting the compound according to formula (IV) with a palladium(0) source and a nucleophile, whereby a compound according to formula (V) is obtained;
Step 2) Providing a compound according to formula (VIb);
Step 3) Reacting the compound according to formula (V) with a compound according to formula (VIb), whereby a compound according to formula (VIIb) is obtained;
Step 4) Subjecting the compound according to formula (VIIb) to an acid induced cleavage from the resin, whereby the compound according to formula (VIIIb) is obtained.

Embodiments of step 1.1.b), step 1.2), step 2), step 3) and/or step 4) are described in detail above. These embodiments can be used for the preparation of the compound according to formula (VIIIb).

In one embodiment, the compound according to formula (VIIIb), a hydrate thereof, solvate thereof, salt thereof, pharmaceutically acceptable salt thereof, or the solvates of salts thereof is prepared as follows:
Step 4) Subjecting the compound according to formula (VIIa) to an acid induced cleavage from the resin, whereby the compound according to formula (VIa) is obtained;
Step 5) Reacting the compound according to formula (VIIIa) with an oxidizing agent, whereby a compound according to formula (VIIIb) is obtained.

Embodiments of step 4) and/or step 5) are described in detail above. These embodiments can be used for the preparation of the compound according to formula (VIIb).

In one embodiment the compound according to formula (VIIIb), a hydrate thereof, solvate thereof, salt thereof, pharmaceutically acceptable salt thereof, or the solvates of salts thereof is prepared as follows:
Step 3) Reacting the compound according to formula (V) with a compound according to formula (VIa), whereby a compound according to formula (VIIa) is obtained;
Step 4) Subjecting the compound according to formula (VIIa) to an acid induced cleavage from the resin, whereby the compound according to formula (VIIIa) is obtained;
Step 5) Reacting the compound according to formula (VIIIa) with an oxidizing agent, whereby a compound according to formula (VIIIb) is obtained.

Embodiments of step 3), step 4) and/or step 5) are described in detail above. These embodiments can be used for the preparation of the compound according to formula (VIIIb).

In one embodiment the compound according to formula (VIIIb), a hydrate thereof, solvate thereof, salt thereof, pharmaceutically acceptable salt thereof, or the solvates of salts thereof is prepared as follows:
Step 1) Providing a compound according to formula (V);
Step 2) Providing a compound according to formula (VIa);
Step 3) Reacting the compound according to formula (V) with a compound according to formula (VIa), whereby a compound according to formula (VIIa) is obtained;
Step 4) Subjecting the compound according to formula (VIIa) to an acid induced cleavage from the resin, whereby the compound according to formula (VIIIa) is obtained;
Step 5) Reacting the compound according to formula (VIIIa) with an oxidizing agent, whereby a compound according to formula (VIIIb) is obtained.

Embodiments of step 1), step 2), step 3), step 4) and/or step 5) are described in detail above. These embodiments can be used for the preparation of the compound according to formula (VIIIb).

In one embodiment the compound according to formula (VIIIb), a hydrate thereof, solvate thereof, salt thereof, pharmaceutically acceptable salt thereof, or the solvates of salts thereof is prepared as follows:
Step 1.1.a) Providing a compound according to formula (IV);
Step 1.2) Reacting the compound according to formula (IV) with a palladium(0) source and a nucleophile, whereby a compound according to formula (V) is obtained;
Step 2) Providing a compound according to formula (VIa);
Step 3) Reacting the compound according to formula (V) with a compound according to formula (VIa), whereby a compound according to formula (VIIa) is obtained;
Step 4) Subjecting the compound according to formula (VIIa) to an acid induced cleavage from the resin, whereby the compound according to formula (VIIIa) is obtained;
Step 5) Reacting the compound according to formula (VIIIa) with an oxidizing agent, whereby a compound according to formula (VIIIb) is obtained.

Embodiments of step 1.1.a), step 1.2), step 2), step 3), step 4) and/or step 5) are described in detail above.

These embodiments can be used for the preparation of the compound according to formula (VIIIb).

In one embodiment the compound according to formula (VIIIb), a hydrate thereof, solvate thereof, salt thereof, pharmaceutically acceptable salt thereof, or the solvates of salts thereof is prepared as follows:
Step 1.1.b) Reacting a compound according to formula (X) with a compound according to formula (III) whereby a compound according to formula (IV) is obtained;
Step 1.2) Reacting the compound according to formula (IV) with a palladium(0) source and a nucleophile, whereby a compound according to formula (V) is obtained;
Step 2) Providing a compound according to formula (VIa);
Step 3) Reacting the compound according to formula (V) with a compound according to formula (VIa), whereby a compound according to formula (VIIa) is obtained;
Step 4) Subjecting the compound according to formula (VIIa) to an acid induced cleavage from the resin, whereby the compound according to formula (VIIa) is obtained;
Step 5) Reacting the compound according to formula (VIIIa) with an oxidizing agent, whereby a compound according to formula (VIIIb) is obtained.

Embodiments of step 1.1.b), step 1.2), step 2), step 3), step 4) and/or step 5) are described in detail above. These embodiments can be used for the preparation of the compound according to formula (VIIIb).

A further aspect of the invention is the use of the compound according to formula (VIIIb) in the process for the preparation of the compound according to formula (I). A further aspect of the invention is the use of the compound according to formula (VIIIb) in the process for the preparation of the compound according to formula (Ia).

A further aspect is the use of the compound according to any one of formulae (III-1), (IV), (VIIa), (VIIb), (VIIIa) and/or (VIIIb) in the process for the preparation of the compound according to formula (I). A further aspect is the use of the compound according to any one of formulae (III-1), (IV), (VIIa), (VIIb), (VIIIa) and/or (VIIIb) in the process for the preparation of the compound according to formula (Ia).

A further aspect is the use of the compound according to any one of formulae (III-1), (IV), (VIIa) and/or (VIIb) in the process for the preparation of the compound according to formula (VIII a) and/or (VIII b).

### EXPERIMENTAL PART

### A. Abbreviations

| | |
|---|---|
| ACN | acetonitrile |
| aq. | aqueous |
| approx. | approximately |
| Boc | tert-butyloxycarbonyl |
| CPME | cyclopentyl methyl ether |
| DAB | (2S)-4-Amino-2-[(tert-butoxycarbonyl)amino]butanoic acid |
| deion. | deionized |
| DEPBT | 3-(diethoxyphosphoryloxy)-1,2,3-benzotriazin-4(3H)-one |
| DIPEA | diisopropylethylamine |
| DIC | N,N′-diisopropylcarbodiimide |
| DMF | N,N-dimethylformamide |
| EDT | Ethanedithiol |
| h | hours |
| IPE | isopropyl ether |
| M | Molar |
| 2-MeTHF | 2-methyltetrahydrofuran |
| min | minute/s |
| Msbh | 4,4′-dimethylsulfinylbenzhydryl |
| N | Normal |
| Oxyma | Ethyl cyanohydroxyiminoacetate |
| Pd | Palladium |
| Pd(PPh₃)₄ | tetrakis(triphenylphosphine)palladium(0) |
| r.t. | room temperature |
| Si-PPZ | SiliaBond ® piperazine |
| TBTU | 2-(1H-Benzotriazole-1-yl)-1,1,3,3-tetramethylaminium |
| | tetrafluoroborate |
| TCEP | tris(2-carboxyethyl)phosphine |
| TEA | triethylamine |
| TFA | trifluoroacetic acid |
| THF | tetrahydrofuran |
| TIS | Triisopropylsilane |

Nomenclature of amino acids and peptide sequences is according to: International Union of Pure and Applied Chemistry and International Union of Biochemistry: Nomenclature and Symbolism for Amino Acids and Peptides (Recommendations 1983). In: Pure & Appl. Chem. 56, Vol. 5, 1984, p. 595-624

### B. General Methods and Reagents

### B.1 HPLC-Methods

### Method 1a:

Equipment: Dionex Ultimate 3000 RS
Column: Waters Aquity BEH 130 C18; 50×2.1 mm; 1.7 μm
Eluent A: 0.05% FA (v/v) in H2O/ACN (99:1 v/v)
Eluent B: 0.05% TFA (v/v) in ACN
Gradient: 0% B— 5 min—100% B—2 min—100% B
Flow rate: 0.4 mL/min Column temperature: 25° C. Detection: 220 nm
Injection volume: 2 μL

### Method 1b:

Equipment: Dionex Ultimate 3000 RS
Column: Waters Aquity BEH 130 C18; 50×2.1 mm; 1.7 μm
Eluent A: 0.05% FA (v/v) in H2O/ACN (99:1 v/v)
Eluent B: 0.05% TFA (v/v) in ACN
Gradient: 20% B—5 min—100% B—2 min—100% B
Flow rate: 0.4 mL/min Column temperature: 25° C. Detection: 220 nm
Injection volume: 2 μL
   Method 2: (see e.g. Example 11)
Equipment: Dionex Ultimate 3000 RS
Stationary phase: C18, 130 Å, 1.7 μm, 2.1×150 mm;
Eluent A: 0.005% (v/v) TFA in H₂O+50 mM NaCl;
Eluent B: 5% (v/v) H₂O in MeOH+50 mM NaCl;
Gradient 50% B—1 min—50% B—20 min—70% B—1 min—90% B—3 min—90% B—0.1 min—50% B—4.9 min—50% B;
Flow rate: 0.3 mL/min, Column temp.: 60° C., Detection UV (220 mu)
   Method 3: (see e.g. Example 15)
Equipment: Knauer 1800 preparative HPLC
Packaging, amount and type: ca. 195 g Kromasil 10MYM C8 (RP)
Bead dimension: D: 50 mm. Prochrom DAC
Flow rate: 76 L/min
Elution pressure: approx. 28 bar
Dection: 210 nm
Temperature: approx. 22° C.
Run time: 53 Min.
Eluent A: water, ACN (9:1) plus 0.1% acetic acid
Eluent B: ACN plus 0.1% acetic acid

| time [min] | flow [mL/min] | % eluent A | % eluent B |
|---|---|---|---|
| 0 | 76 | 80 | 20 |
| 11 | 76 | 66 | 34 |
| 26 | 76 | 73 | 27 |
| 33 | 76 | 0 | 100 |
| 43 | 76 | 0 | 100 |
| 43.1 | 76 | 89 | 11 |
| 54 | 76 | 89 | 11 |

### B.2 Reagents

40 kDa methoxy poly(ethylene glycol) maleimido propionamide (linear 40k mPEG maleimide); CAS No 724722-89-8; From Dr. Reddys Inc., Lot No 233101301; Weight average molecular weight, Mw (GPC) 40500 Da Polydispersity (GPC) 1.08.

### C. Examples

### Example 1

### Allyl-N-(tert-butoxycarbonyl)-O-[(4-nitrophenoxy)carbonyl]-L-tyrosinate

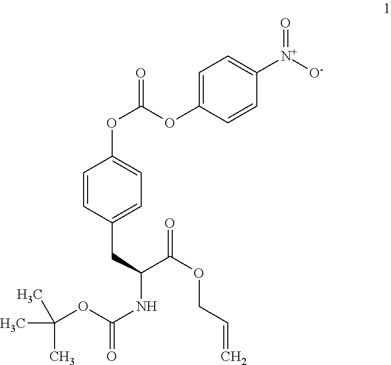 [SMILES: C=CCOC(=O)[C@H](Cc1ccc(OC(=O)Oc2ccc([N+](=O)[O-])cc2)cc1)NC(=O)OC(C)(C)C]

115 g (1.5 eq.) 4-Nitrophenyl chloroformate were dissolved in 570 mL 2-MeTHF and the solution was cooled to 0° C. 122 g (1 eq.) N-Boc-tyrosine allyl ester was dissolved in 2-MeTHF and 1.2 eq TEA was added. The resulting solution was added slowly to the chloroformate solution at 0° C. over an extended period of time. The reaction mixture was stirred first at 0° for 75 min and then at room temperature for 120 min. The reaction mixture was quenched by addition of water. The organic layer was washed with 5% NaHCO₃ solution and with water. The organic layer was concentrated at reduced pressure to half of the original volume. 2-MeTHF was added and evaporated to azeotropically remove residual water. The product was precipitated at 0° C. using IPE as antisolvent, collected by filtration and washed with IPE. The filter cake was dried in vacuo for 3 h at 35° C. followed by 20h at room temperature.
Yield: 85%
Purity (method 1b): 97.6% (Rt=5.07 min)

### Example 2

### ((2S)-4-{[(4-{(2S)-3-(allyloxy)-2-[(tert-butoxycarbonyl)amino]-3-oxopropyl}phenoxy)carbonyl]amino}-2-[(tert-butoxycarbonyl)amino]butanoic acid

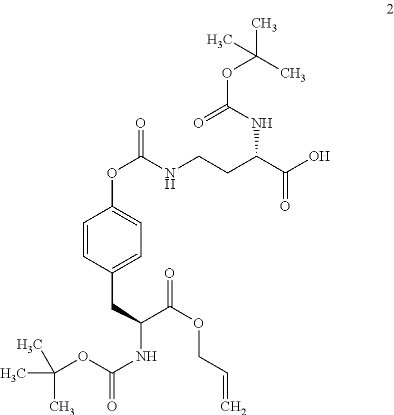 [SMILES: C=CCOC(=O)[C@H](Cc1ccc(OC(=O)NCC[C@H](NC(=O)OC(C)(C)C)C(=O)O)cc1)NC(=O)OC(C)(C)C]

20.0 g (41.1 mmol) Example 1 were suspended in 80 mL MeTHF and 13.5 g (61.7 mmol, 1.5 eq.) Boc-Dab-OH and 80 mL deion. water were added. After cooling to 0-5° C. with an ice-bath 10.6 mL (61.7 mmol, 1.5 eq.) DIPEA were added and the resulting mixture was stirred for 4.5 h at 0-5° C. 100 mL cyclohexane, 10 mL acetone and 80 mL deion. water were added to the reaction mixture and after separation of the layers, the aqueous layer was extracted 5× with 120 mL of a 5:1 mixture of IPE and acetone. 250 mL MeTHF were added to the aqueous layer and it was acidified to pH approx. 1 using 18% HCl (aq.). The aqueous layer was discarded and the organic layer was washed with 3×50 mL deion. water. The organic layer was concentrated and 2×100 mL MeTHF were added and evaporated to azeotropically remove residual water yielding 19.5 g (34.5 mmol) of Example 2 after drying for 16 h at r.t. under reduced pressure.

### Example 3: Amidation

### 9H-fluoren-9-ylmethyl [(2R)-1-({bis[4-(methylsulfinyl)phenyl]methyl}sulfanyl)-3-oxobutan-2-yl]carbamate

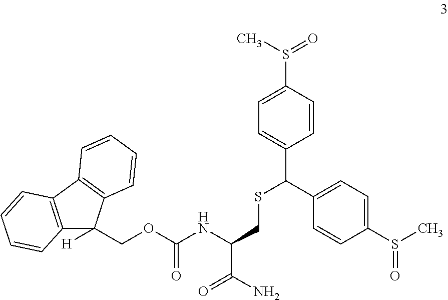 [SMILES: CS(=O)c1ccc(C(SC[C@H](NC(=O)OCC2c3ccccc3-c3ccccc32)C(N)=O)c2ccc(S(C)=O)cc2)cc1]

1 eq. (2R)-3-[bis(4-methylsulfinylphenyl)methylsulfanyl]-2-(9H-fluoren-9-yloxycarbonylamino)propanoic acid 3a

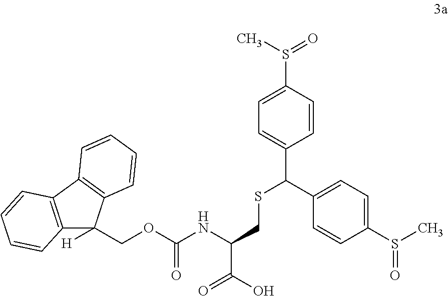 [SMILES: CS(=O)c1ccc(C(SC[C@H](NC(=O)OCC2c3ccccc3-c3ccccc32)C(=O)O)c2ccc(S(C)=O)cc2)cc1]

was dissolved in THF and the solution was cooled to 0° C. 2.5 eq. DEPBT, 2.5 eq. DIPEA and 3 eq. ammonia solution 25% were added. The reaction was stirred at 0° C. for 30 min, then at ambient temperature for 330 min. The solvent was evaporated and 2-MeTHF and 5% NaHCO₃ solution were added. The organic layer was washed repeatedly with 5% NaHCO₃ solution and with 1 N HCl. The organic layer was concentrated and taken up in HF. Example 3 was precipitated using IPE as antisolvent. The precipitate was filtered, washed with IPE and dried at r.t. under reduced pressure.
Yield: 93%
Purity (HPLC-method 1b): 98.1% (Rt=3.45 min)
LC-MS (method 1b): m/z=633 (M+H)+

### Example 4

### S-{bis[4-(methylsulfinyl)phenyl]methyl}-L-cysteine)

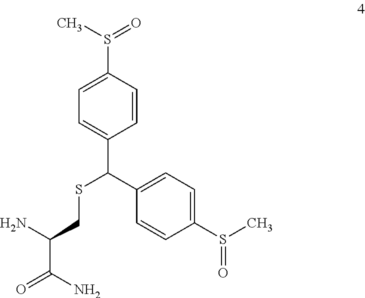 [SMILES: CS(=O)c1ccc(C(SC[C@H](N)C(N)=O)c2ccc(S(C)=O)cc2)cc1]

278.4 g of Example 3 was dissolved in 1 L DMF and 350 g SiliaBond® piperazine (Si-PPZ) were added at 20° C. The suspension was incubated for 44 h and filtered to remove the silica. The filtrate was used directly in the synthesis of the Example 5.
Purity (method 1a): 96.5% (Rt=7.71 min)
LC-MS (method 1a): m/z=411 (M+H)+

### Example 5

### O1-allyl O4-tert-butyl (2R)-2-[[4-[[(3S)-4-[[(1R)-1-[bis(4-methylsulfinylphenyl)methylsulfanylmethyl]-2-oxo-propyl]amino]-3-(tert-butoxycarbonylamino)-4-oxo-butyl]carbamoyloxy]phenyl]methyl]butanedioate

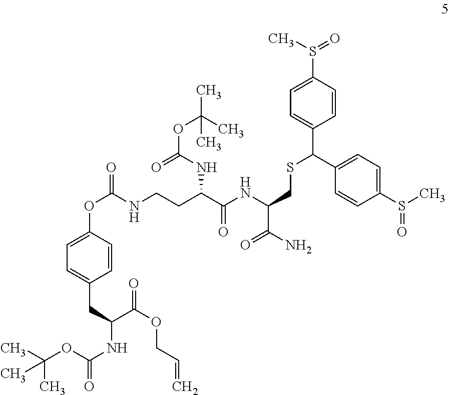 [SMILES: C=CCOC(=O)[C@H](Cc1ccc(OC(=O)NCC[C@H](NC(=O)OC(C)(C)C)C(=O)N[C@@H](CSC(c2ccc(S(C)=O)cc2)c2ccc(S(C)=O)cc2)C(N)=O)cc1)NC(=O)OC(C)(C)C]

1 eq. of Example 2 was dissolved in DMF and was cooled to 0-5° C. 1.0 eq. TBTU and 3 eq. DIPEA were added and the solution of Example 4 obtained as described above (approx. 1.2 eq.) was slowly added. After stirring for 15 min at 0-5° C. cooling was removed and the reaction mixture was stirred for 1 h at r.t. Then 2-MeTHF was added to the reaction mixture and it was extracted with a mixture of brine/deion. water 1:1, followed by 3×1N HCl and 3× deion. water. The organic layer was concentrated and 2×2-MeTHF were added and evaporated to azeotropically remove residual water yielding a residue The residue was dissolved in 2-MeTHF at 35° C. and the resulting solution was added dropwise to CPME at 0-5° C. while stirring. After stirring for an additional 10 min the precipitate was filtered off and was washed with 3×CPME. Example 5 was dried for 2 h at 35° C. under reduced pressure. The obtained product was dissolved in 2-MeTHF at 35° C. and the resulting solution was added dropwise to CPME at 0-5° C. while stirring. After stirring for an additional 10 min the precipitate was filtered off and was washed with 3×100 mL CPME. After a short drying for 30 min at 35° C. under reduced pressure, the obtained product was dissolved in 2-MeTHF at 35° C. and the resulting solution was added dropwise to CPME at 0-5° C. while stirring. After stirring for an additional 10 min the precipitate was filtered off and was washed 3× with CPME. The obtained product was dried at 35° C. for 16 h under reduced pressure yielding Example 5.
Yield: 79%
Purity (method 1a): 98.5% (Rt=4.52 min)
LC-MS (method 1a): m/z=958 (M+H)+

### Example 6

### (2R)-2-[[4-[[(3S)-4-[[(1R)-1-[bis(4-methylsulfinylphenyl)methylsulfanylmethyl]-2-oxo-propyl]amino]-3-(tert-butoxycarbonylamino)-4-oxo-butyl]carbamoyloxy]phenyl]methyl]-4-tert-butoxy-4-oxo-butanoic acid

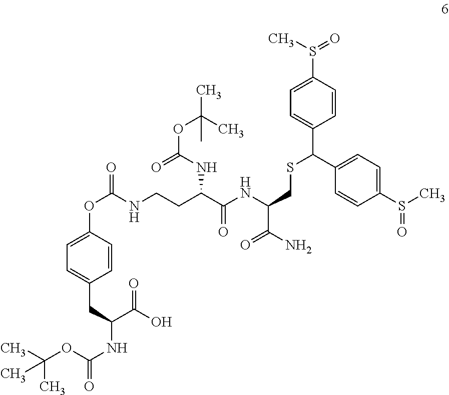 [SMILES: CS(=O)c1ccc(C(SC[C@H](NC(=O)[C@H](CCNC(=O)Oc2ccc(C[C@H](NC(=O)OC(C)(C)C)C(=O)O)cc2)NC(=O)OC(C)(C)C)C(N)=O)c2ccc(S(C)=O)cc2)cc1]

1 eq. Example 5 was dissolved in THF and was cooled to 0-5° C. 5 eq. formic acid, 5 eq. TEA and 0.03 eq. Pd(PPh₃)₄ were added. After stirring for 6 h at r.t. 5% NaHCO₃ (aq.) was added and the mixture was extracted with 3×2-MeTHF·2-MeTHF was added to the aqueous layer and it was acidified with 18% HCl (aq.) to pH of approx. 1. The aqueous layer was discarded and the organic layer was washed 5× with deion. water. The organic layer was concentrated and 3×2-MeTHF was added and evaporated to azeotropically remove residual water, yielding a residue. The residue was dissolved in THF and was added dropwise IPE while stirring. The precipitate was filtered off and washed with 1×IPE, 2×2-MeTHF and 1×IPE. The obtained filter cake was dried at 30° C. under reduced pressure for 64 h.
Yield: 78%
Purity (method 1b): 98.9% (Rf=3.11 min), white powder
LC-MS (method 1b): m/z=918 (M+H)+

### Example 7a

### Tentagel™ Based Amide Resin Bound ADM (2-52) [Linear]

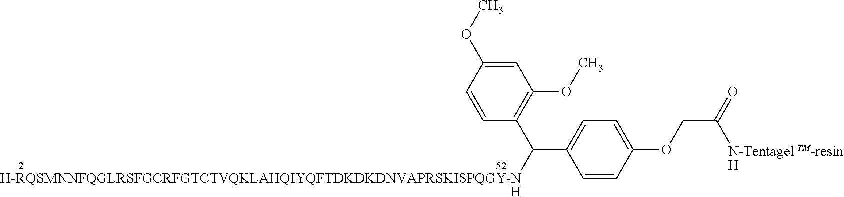

The peptide was assembled stepwise on a Tentagel™ based amide resin on an automated peptide synthesizer (Protein Technologies Inc. Symphony). 8 poly-propylene reaction vessels were used in parallel preforming the identical chemistry. Each vessel was loaded with 0.05 mmol Tentagel™ based Rink resin for a total batch size of 0.4 mmol.

Each amino acid is added in 8 fold molar access with regard to the loading of the resin. The amino acids were Fmoc protected as the N-terminal protecting group and the protecting groups indicated below were used for side chain functionalities. Also 188 mg (0.59 mmol, 7.8 eq.) TBTU and 0.21 ml (1.2 mmol, 16 eq.) DIEA were added. Reactions were performed in DMF as solvent, whereas DMF was used in an amount sufficient to swell the resin and agitate it freely. Reaction time per amino acid was approx. 1 hour.

Cleavage of the Fmoc protecting groups was achieved using 20% piperidine/DMF, whereas 20% piperidine/DMF was used in an amount sufficient to swell the resin and agitate it freely.

The coupling sequence was as follows:
1. Tyr(tBu) (Tyr=Y=AA 52 of human ADM)
2. Gly (Gly=G=AA 51 of human ADM)
3. Gln(Trt) (Gn=Q=AA 50 of human ADM)
4. Pro (Pro=P=AA 49 of human ADM)
5. Ser(tBu) (Ser=S=AA 48 of human ADM)
6. Ile (Ile=I=AA 47 of human ADM)
7. Lys(Boc) (Lys=K=AA 46 of human ADM)
8. Ser(tBu) (Ser=S=AA 45 of human ADM)
9. Arg(pbf) (Arg=R=AA 44 of human ADM)
10. Pro (Pro=P=AA 43 of human ADM)
11. Ala (Ala=A=AA 42 of human ADM)
12. Val (Val=V=AA 41 of human ADM)
13. Asn(Trt) (Asn=N=AA 40 of human ADM)
14. Asp(OtBu) (Asp=D=AA 39 of human ADM)
15. Lys(Boc) (Lys=K=AA 38 of human ADM)
16. Asp(OtBu) (Asp=D=AA 37 of human ADM)
17. Lys(Boc) (Lys=K=AA 36 of human ADM)
18. Asp(OtBu) (Asp=D=AA 35 of human ADM)
19. Thr(tBu) (hr=T=AA 34 of human ADM)
20. Phe (Phe=F=AA 33 of human ADM)
21. Gln(Trt) (Gn=Q=AA 32 of human ADM)
22. Tyr(tBu) (Tyr=Y=AA 31 of human ADM)
23. Ile (Ile=I=AA 30 of human ADM)
24. Gln(Trt) (Gln=Q=AA 29 of human ADM)
25. His(Trt) (His=H=AA 28 of human ADM)
26. Ala (Ala=A=AA 27 of human ADM)
27. Leu (Leu=L=AA 26 of human ADM)
28. Lys(Boc) (Lys=K=AA 25 of human ADM)
29. Gln(Trt) (Gln=Q=AA 24 of human ADM)
30. Val (Val=V=AA 23 of human ADM)
31. Thr(tBu) (hr=T=AA 22 of human ADM)
32. Cys(Trt) (Cys=C=AA 21 of human ADM)
33. Thr(tBu) (hr=T=AA 20 of human ADM)
34. Gly (Gly=G=AA 19 of human ADM)
35. Phe (Phe=F=AA 18 of human ADM)
36. Arg(pbf) (Arg=R=AA 17 of human ADM)
37. Cys(Acm) (Cys=C=AA16 of human ADM)
38. Gly (Gly=G=AA 15 of human ADM)
39. Phe (Phe=F=AA 14 of human ADM)
40. Ser(tBu) (Ser=S=AA 13 of human ADM)
41. Arg(pbf) (Arg=R=AA 12 of human ADM)
42. Leu (Leu=L=AA 11 of human ADM)
43. Gly (Gly=G=AA 10 of human ADM)
44. Gln(Trt) (Gln=Q=AA 9 of human ADM)
45. Phe (Phe=F=AA 8 of human ADM)
46. Asn(Trt) (Asn=N=AA 7 of human ADM)
47. Asn(Trt) (Asn=N=AA 6 of human ADM)
48. Met (Met=M=AA 5 of human ADM)
49. Ser(tBu) (Ser=S=AA 4 of human ADM)
50. Gln(Trt) (Gln=Q=AA 3 of human ADM)
51. Arg(pbf) (Arg=R=AA 2 of human ADM)

### Example 7b

### Tentagel™ Based Amide Resin Bound ADM (2-52) [Cyclic]

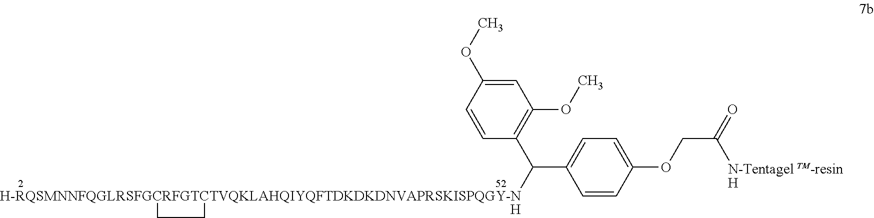

On-resin oxidation of Example 7a was achieved using Cys(Trt) and Cys(Acm) protection with concomitant cleavage of protecting groups and oxidation to a disulfide bond using Iodine (8 equivalents of Iodine plus 8 equivalents of DIEA with a reaction time of 30 minutes). Oxidation was confirmed by sample cleavage and analysis using HPLC and MALDI-MS.

### Example 8a

### Coupling Reaction, Preparation of Resin-Bound Example 8a

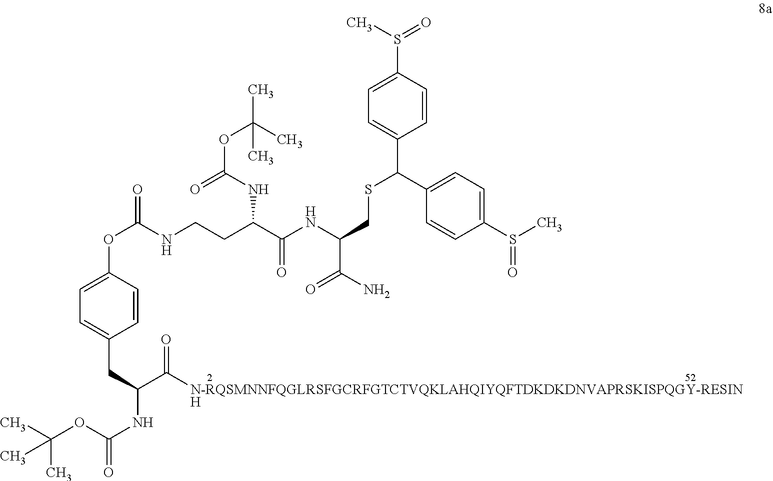

In Example 8a above, the Tentagel™ based amide resin bound to the ADM (2-52) is abbreviated as “resin”. Example 6 and Tentagel™ based amide resin bound ADM (2-52) according to Example 7a were coupled to obtain resin bound Example 8a. Coupling reactions were executed either with DIC/OxymaPure®, or DEPBT/DIPEA with appropriate coupling (1.5-24.0 h) and Fmoc deprotection (0.25-0.5 h) times at ambient pressure and temperature. 20% piperidine in DMF was used for Fmoc deprotection. After coupling steps, an acetylation was optionally performed using acetic anhydride During amino acid addition cycles, DMF and IPA were used as solvents for the washing steps after acetylation or Fmoc deprotection. DMF, IPA and ACN were used in washing steps at the end of the SPPS.

### Example 8b

### Coupling Reaction, Preparation of Resin-Bound Example 8b

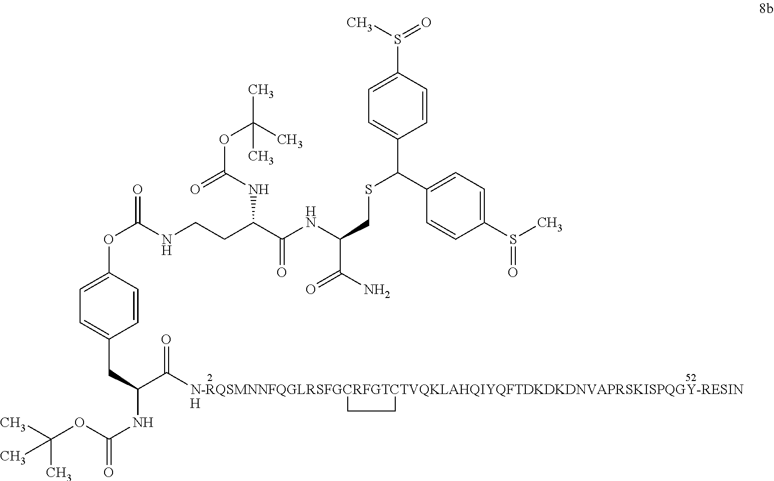

In Example 8b above, the Tentagel™ based amide resin bound to the ADM (2-52) is abbreviated as “resin”. Example 6 and Tentagel™ based amide resin bound ADM (2-52) according to Example 7b were coupled to obtain resin bound Example 8b. Coupling reactions were executed either with DIC/OxymaPure®, or DEPBT/DIPEA with appropriate coupling (1.5-24.0 h) and Fmoc deprotection (0.25-0.5 h) times at ambient pressure and temperature. 20% piperidine in DMF was used for Fmoc deprotection. After coupling steps, an acetylation was optionally performed using acetic anhydride During amino acid addition cycles, DMF and IPA were used as solvents for the washing steps after acetylation or Fmoc deprotection. DMF, IPA and ACN were used in washing steps at the end of the SPPS.

### Example 9a

### Cleavage from the Resin

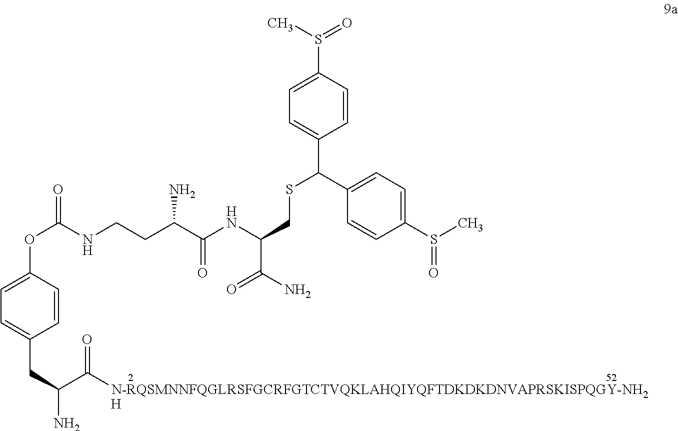

Resin bound Example 8a was cleaved from the resin by adding said Example 8a to a cleavage cocktail of TFA/EDT/H₂O/TIS (90.0:5.0:2.5:2.5) at 25° C. After incubation for 3.5 h, the cleavage cocktail was separated from the resin, the resin was washed with TFA and cleavage cocktail and TFA washes were pooled. The product was precipitated by adding IPE to the pool at −10° C. The suspension was incubated at 0° C. for 30 min, the precipitate filtered off, washed with IPE and dried at reduced pressure (<5 mbar) and ambient temperature to yield the crude linear product Example 9a.

### Example 9b

### Cleavage from the Resin

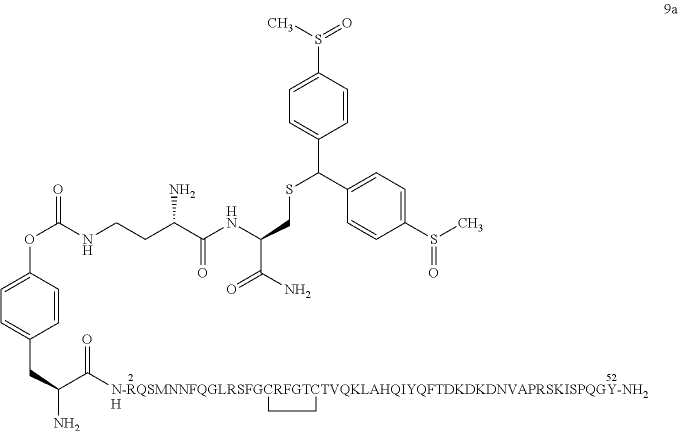

Resin bound Example 8b was cleaved from the resin by adding said Example 8b to a cleavage cocktail of TFA/EDT/H₂O/TIS (90.0:5.0:2.5:2.5) at 25° C. After incubation for 3.5 h, the cleavage cocktail was separated from the resin, the resin was washed with TFA and cleavage cocktail and TFA washes were pooled. The product was precipitated by adding IPE to the pool at −10° C. The suspension was incubated at 0° C. for 30 min, the precipitate filtered off, washed with IPE and dried at reduced pressure (<5 mbar) and ambient temperature to yield the crude linear product Example 9b.

### Example 10: Purification of Example 9a

Crude linear Example 9a was dissolved in 0.1% TFA+3% ACN aq. Optionally, 5% TCEP (w/w with regard to the crude peptide) was added and the solution incubated for 60 min at ambient temperature. C8-bonded silica (pore size: 100 Å, particle size: 10 μm) was used as stationary phase. The column was pre-equilibrated with eluent A (0.1% TFA+3% ACN aq.), the crude peptide loaded and the column shortly rinsed with 0.1% TFA+3% ACN aq. Elution was effected with a gradient of 18% to 100% eluent B (0.1% TFA+80% ACN aq.) in 165 min. Fractions were collected and pooled based on analytic HPLC results.

### Example 11: Oxidation

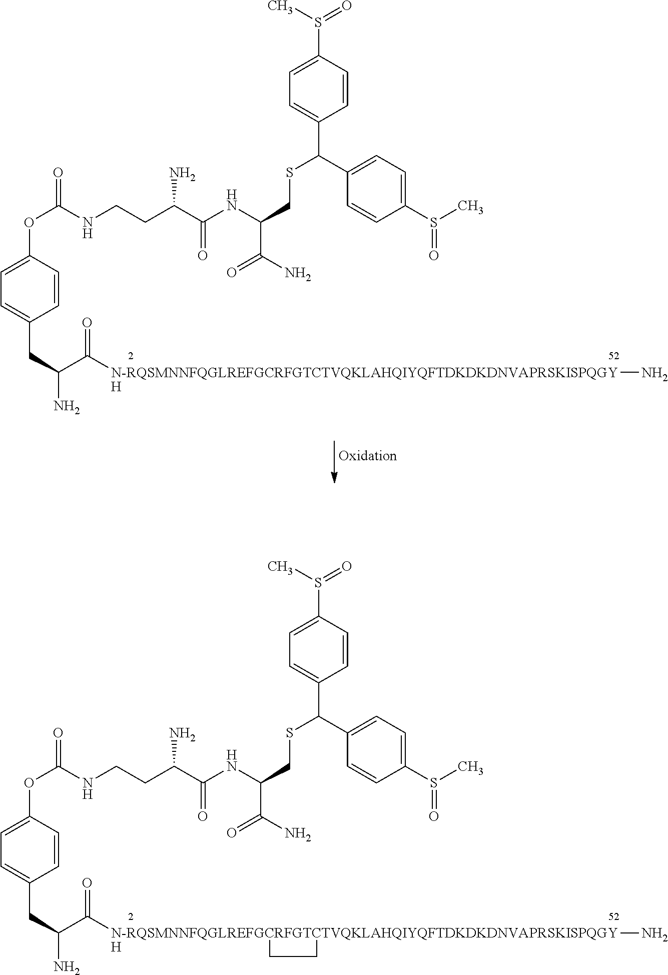

The fraction pool of Example 10 was diluted with an equal volume of water. A reagent solution consisting of 0.4 M I₂ and 1.2 M KI in water was added slowly while stirring until the solution remained colored The reaction mixture was stirred for ≥5 n in and the oxidation solution was analyzed for completion of the reaction by analytic HPLC (method 2 in section B.1). Ascorbic acid was added to the solution until it turned colorless and filtered optionally. The solution was applied to the next purification stage.

### Example 12: Purification of Example 11

Two dimensional HPLC purification of Example 11 was performed using C8-bonded silica (pore size: 100 Å, particle size: 10 μm) as stationary phase. The stationary phase was pre-equilibrated before each run with 0.1% TFA+3% ACN aq., the analyte solution was loaded and the column and shortly rinsed with 0.1% H₃PO₄+3% ACN aq.

Eluents A and B in the first dimension were 0.1% H₃PO₄+3% ACN aq. and 0.1% H₃PO₄+80% ACN aq., respectively. After flushing with eluent A, elution was effected by a linear gradient of 18 to 100% in the concentration of eluent B within 165 min. Fractions were collected and pooled based on analytic HPLC results. The pool was diluted 1:1 with water and was subjected to the next purification step.

Eluents A and B in the second dimension were 0.1% TFA+3% ACN aq. and 0.1% TFA+80% ACN aq., respectively. After flushing with eluent A, elution was effected by a linear gradient of 18 to 100% in the concentration of eluent B within 165 min. Fractions were collected and pooled based on analytic HPLC results. The pool was diluted 1:1 with water and subjected to lyophilization. The lyophilized sublots were then dissolved in 0.1% TFA, subjected to microfiltration, and lyophilized again.

### Example 13: Deprotection

### O-{[(3S)-3-Amino-4-{[(2R)-1-amino-1-oxo-3-sulfanylpropan-2-yl]amino}-4-oxobutyl]-carbamoyl}-L-tyrosyl-adrenomedullin (2-52)

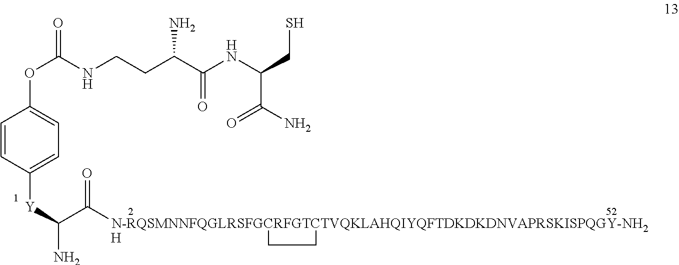

3.0 g Example 11 was added to a mixture of 46 ml trifluoroacetic acid and 4 ml triisopropyl silane at a temperature of −5° C. with stirring in an inert atmosphere (Argon). 0.3 g tetrabutylammonium iodide were added and stirring was continued for 4.5 hours at 0° C. at 100 rpm stirring speed. Subsequently, 125 ml of pre-cooled (−10° C.) diisopropylether were added within 30 min while maintaining the temperature between −5° C. and 5° C. and increased stirring speed. Stirring was continued for another 30 min and the suspension is filtered. The filter cake was washed with diisopropylether (3×30 ml), and dried at 2 mbar and ambient temperature (22-24° C.) for 18 h.
Yield: 2.86 g (off-white powder), 100% yield without correction for assay or purity (85.2 area % HPLC.

### Example 14: PEGylation of Example 13

### O-{[(3S)-3-Amino-4-({(2R)-1-amino-3-[(2,5-dioxo-1-{3-oxo-3-[(2-{ω-methoxy-poly-oxyethylen[40 kDa]}ethyl)amino]propyl}pyrrolidin-3-yl)sulfanyl]-1-oxopropan-2-yl}amino)-4-oxobutyl]carbamoyl}-L-tyrosyl-adrenomedullin (2-52)(“PEG-ADM”)

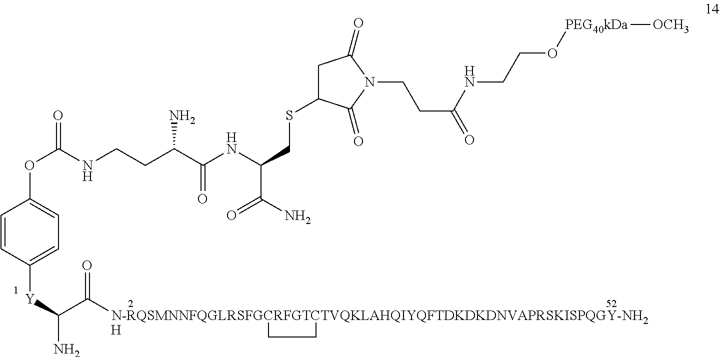

20.3 g (0.462 mmol, corrected for assay) of 40 kDa-mPEG-Maleimide were dissolved in a mixture of 400 g sodium acetate buffer (50 mM, pH 3.5) and 47.2 g acetonitrile with stirring at ambient temperature (22-24° C.). 2.0 g (0.229 mmol, corrected for assay and purity) Example 13 was added and stirring was continued at room temperature for another 3 h. The resulting solution (470 ml total volume; purity by HPLC: 81.6 area %; c=2.08 mg/ml ADM-payload; 977 mg ADM-payload (˜68% yield)) was stored −20° C. until further use. It could directly be subjected to the following purification by preparative HPLC as described in Example 15

### Example 15: Purification of Example 14

The solution from the previously described conjugation experiment (Example 14, “PEG-ADM”) was filtered through a G4 glass frit and 100 ml of the available solution (208 mg ADM-payload) were purified by preparative HPLC (2×50 ml injections), see method 3 in section general methods. Purification and fractioning were performed according to method 2 described in the section HPLC-methos above. The fractions containing the purified product were collected and combined. A total of 770 ml eluate containing purified product was collected. A yield of 350 ml with 0.27 mg/ml ADM-payload and 420 ml with 0.25 mg/ml ADM-payload was achieved (202 mg (97%) ADM-payload overall).

### Example 16: Crossflow Filtration

770 ml PEG-ADM eluate solution (202 mg ADM-payload; Example 15) from the HPLC purification according to Example 15 were subjected to a concentration and buffer exchange via crossflow filtration using a 0.2 m2 10 kDa Hydrosart® membrane slice cassette (Sartorius). After the addition of 292 g citrate buffer (6 g citric acid monohydrate, 2.28 g sodium hydroxide, 1005 g water, adjusted to pH 4 with hydrochloric acid) the total volume of the retentate was reduced to 70 ml (transmembrane pressure: ˜1 bar). Afterwards a ten-fold exchange of the volume with the same citric acid buffer was performed and finally the retentate is further concentrated to 50 ml. The retentate was drained and all pipes and membrane are flushed with citric acid buffer. The retentate and the rinse fractions were combined. 101 g PEG-ADM (cf. compound according to example 13 or compound according to formula (Ia)) solutions is recovered (2 mg/ml ADM-payload).

1. A process for the preparation of a compound according to formula (I) 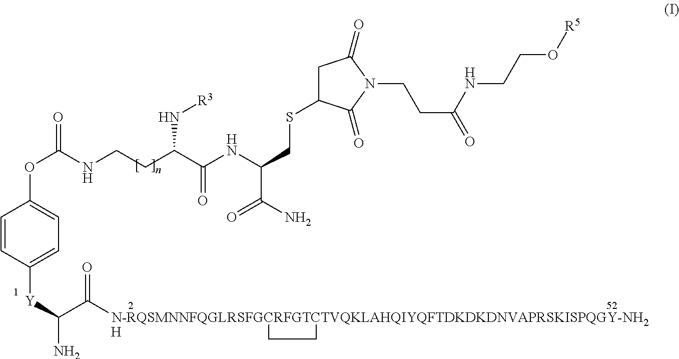
a hydrate thereof, solvate thereof, salt thereof, pharmaceutically acceptable salt thereof, or the solvates of salts thereof, comprising the following steps:
Step 1) Providing a compound according to formula (V) 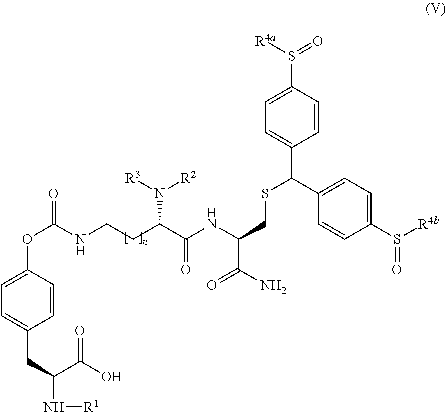
Step 2) Providing a compound according to formula (VIa) or according to formula (VIb) 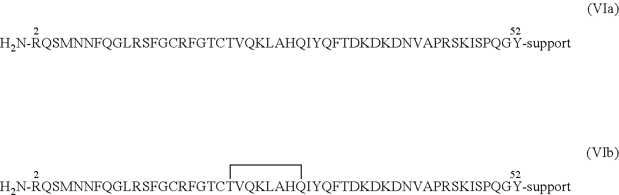
Step 3) Reacting the compound according to formula (V) with a compound according to formula (VIa) or (VIb), whereby a compound according to formula (VIIa) or (VIIb) 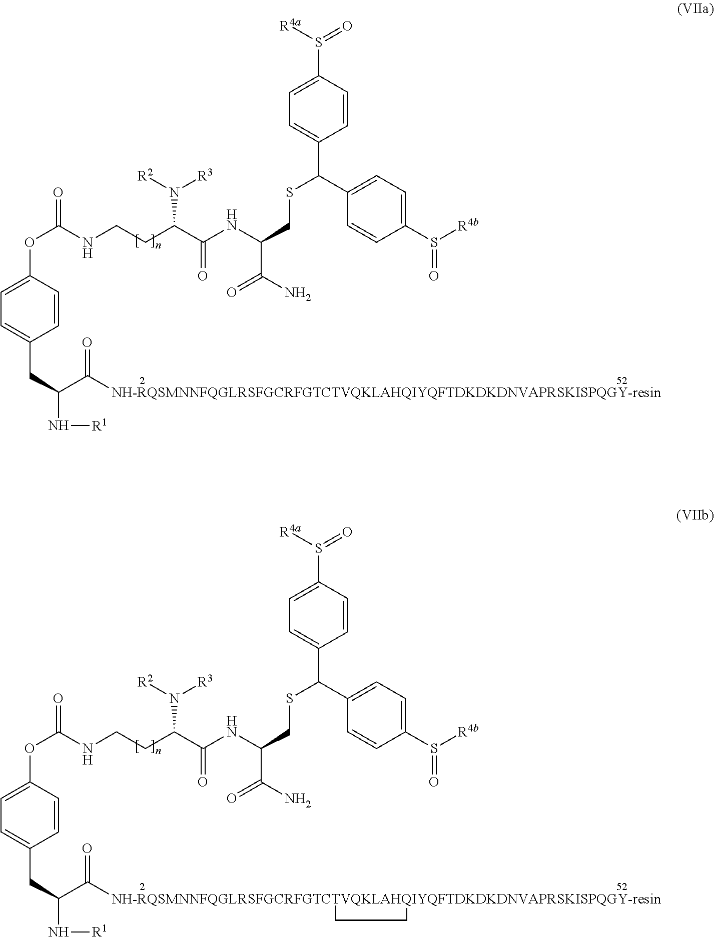
is obtained;
Step 4) Subjecting the compound according to formula (VIIa) or formula (VIIb) obtained in step 3) to an acid induced cleavage from the resin, whereby a compound according to formula (VIIIa) or (VIIIb) 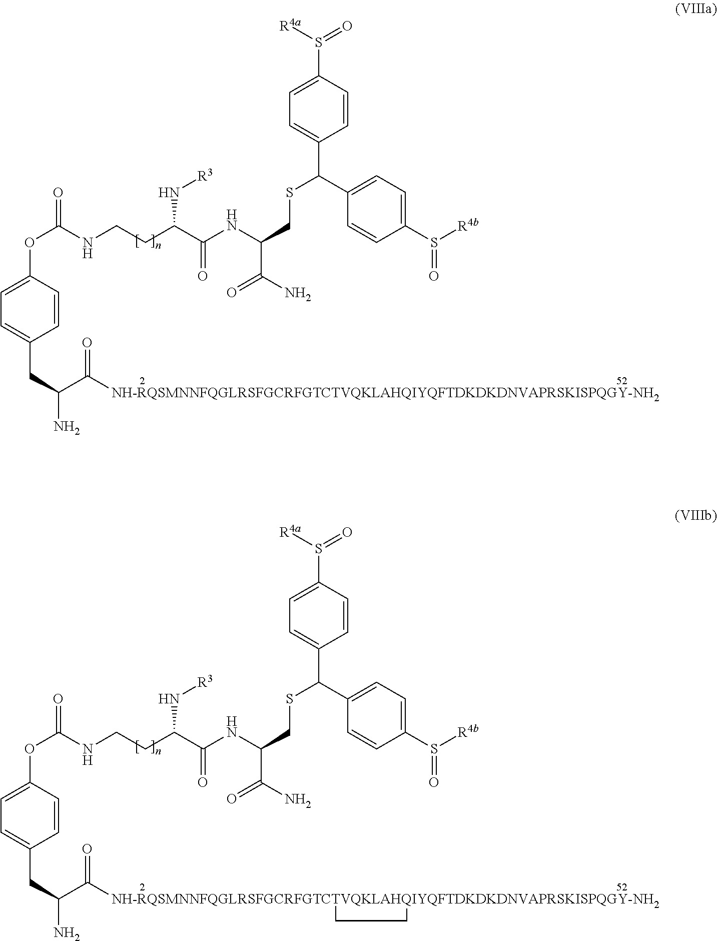
is obtained, wherein
if the compound according to formula (VIIa) or formula (VIIb) comprises as R¹ a (9H-fluoren-9-ylmethoxy)carbonyl group, said compound is subjected to a base-induced cleavage of the (9H-fluoren-9-ylmethoxy)carbonyl group and then to the acid induced cleavage from the resin, whereby a compound according to formula (VIIIa) or (VIIIb) is obtained;
Step 5) Optionally, if the product of step 4) is a compound according to formula (VIIIa), said compound is subjected to reaction with an oxidizing agent, whereby a compound according to formula (VIIIb) is obtained;
Step 6) Reacting the compound according to formula (VIIIb) obtained in step 4) or step 5) with a cleavage cocktail, whereby a compound according to formula (II) 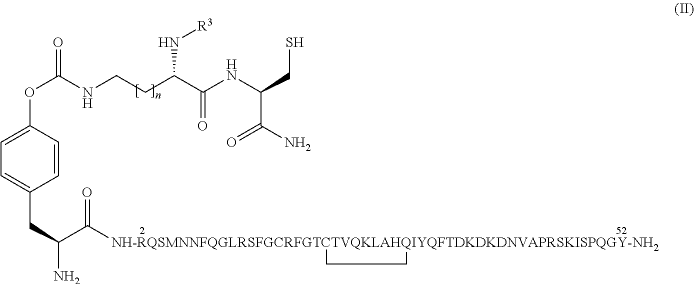
is obtained;
Step 7) Reacting the compound according to formula (II) obtained in step 6) with a compound according to the formula (IX) 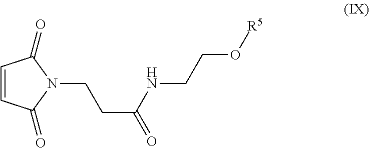
whereby the compound according to formula (I) is obtained;
in which
n represents the number 0, 1 or 2,
R¹ represents tert-butyloxycarbonyl or (9H-fluoren-9-ylmethoxy)carbonyl,
R² represents tert-butyloxycarbonyl,
R³ represents hydrogen, methyl, ethyl, n-propyl or isopropyl,
R^{4a} represents methyl, ethyl, n-propyl, isopropyl or butyl,
R^{4b} represents methyl, ethyl, n-propyl, isopropyl or butyl,
R⁵ represents a linear or branched PEG 20 kDa to 80 kDa endcapped with a methoxy-group.

2. The process according to claim 1, wherein the compound according to formula (V) provided in step 1) is prepared by the following process comprising the steps:
Step 1.1.a): Providing a compound according to formula (IV) 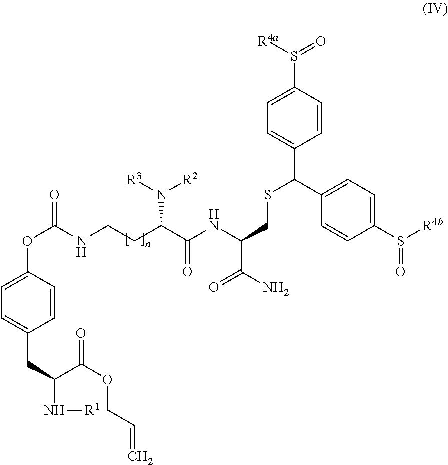
Step 1.2): Reacting the compound according to formula (IV) with a palladium(0) source and a nucleophile, whereby a compound according to formula (V) is obtained.

3. The process according to claim 1, wherein the compound according to formula (V) provided in step 1) is prepared by the following process comprising the steps:
Step 1.1.b): Reacting a compound according to formula (X) 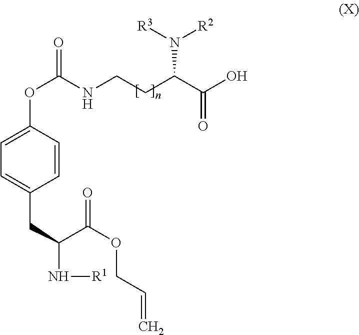
with a compound according to formula (III) 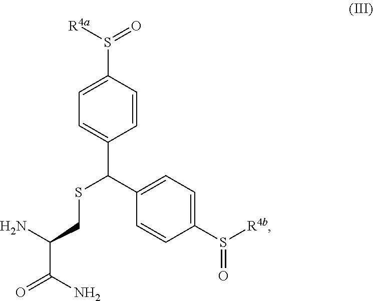
whereby a compound according to formula (IV) is obtained;
Step 1.2): Reacting the compound according to formula (IV) with a palladium(0) source and a nucleophile, whereby a compound according to formula (V) is obtained.

4. The process according to claim 1, wherein the compound according to formula (III) is the compound according to formula (III-1)

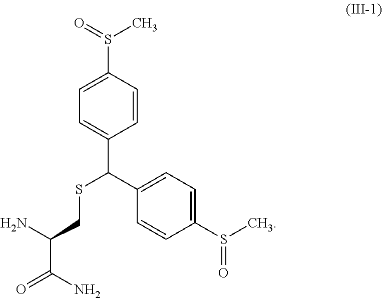 [SMILES: CS(=O)c1ccc(C(SC[C@H](N)C(N)=O)c2ccc(S(C)=O)cc2)cc1]

5. The process according to claim 2, the palladium(0) source in step 1.2) is selected from the group consisting of tetrakis(triphenylphosphine)palladium(0) ((Pd(PPh₃)₄)), palladium(II)bis(triphenylphosphine) dichloride (PdCl₂(Ph₃P)₂), palladium charcoal (PD/C), palladium(II)-acetate (Pd(OAc)₂) and mixtures thereof.

6. The process according to claim 1, wherein step 3) is conducted in the presence of a coupling reagent.

7. The process according to claim 1, wherein the acid for the acid induced cleavage in step 4) is selected from trifluoroacetic acid, hydrogen chloride, hydrogen chloride in dioxane and mixtures thereof.

8. The process according to claim 1, wherein the oxidizing agent in step 5) is selected from iodine, iodine salts, oxygen, H₂02, dipyridyl disulfide (DPDS) and mixtures thereof.

9. The process according to claim 1, wherein the cleavage cocktail in step 6) comprises trifluoroacetic acid, ammonium iodides, and/or mixtures thereof.

10. The process according to claim 1, wherein the compound according to formula I is the compound according to formula (Ia)

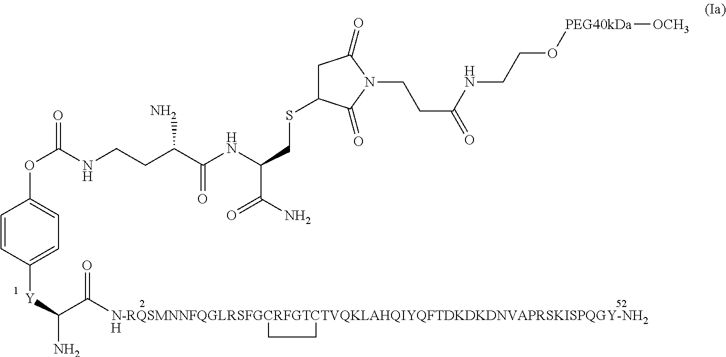

11. A compound selected from the compounds according to any one of formulae (IV), (VIIa), (VIIb), (VIIIa) and/or (VIIIb) 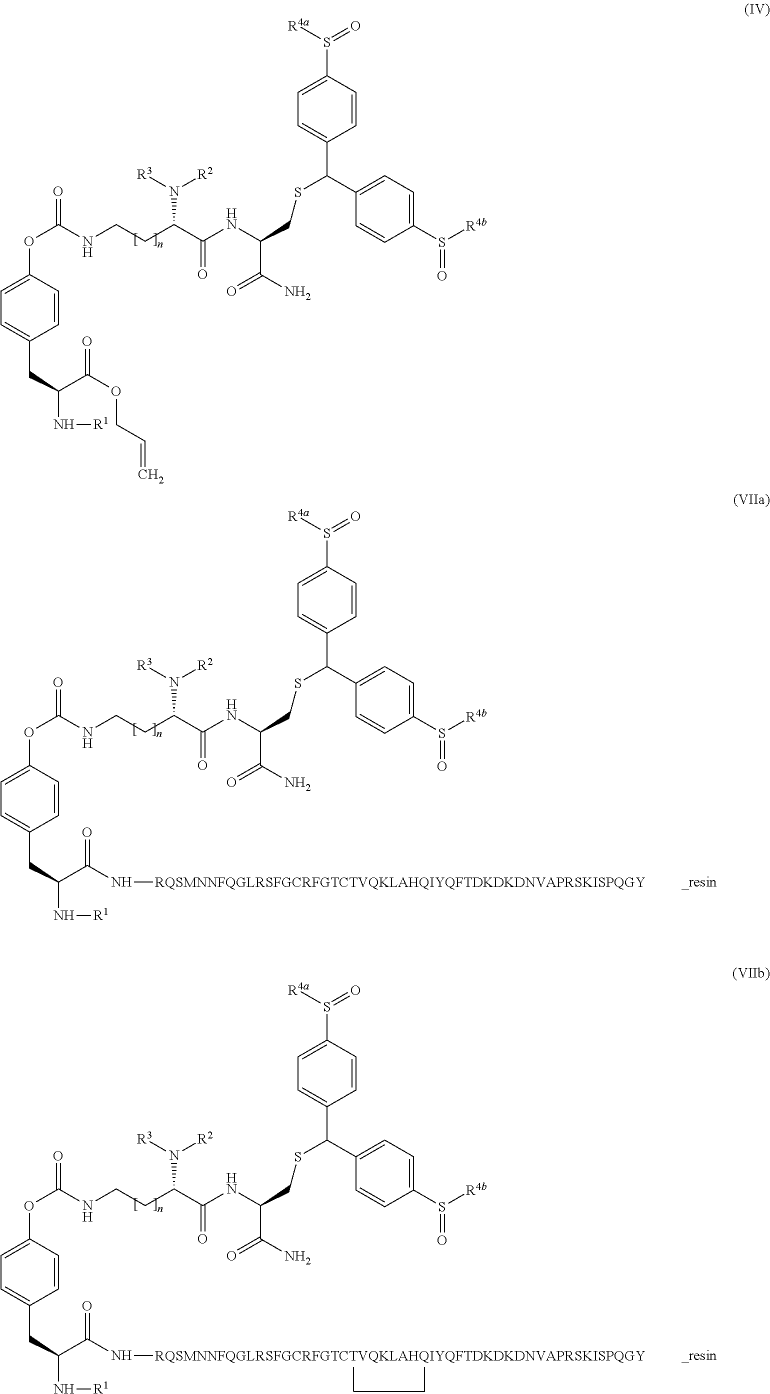 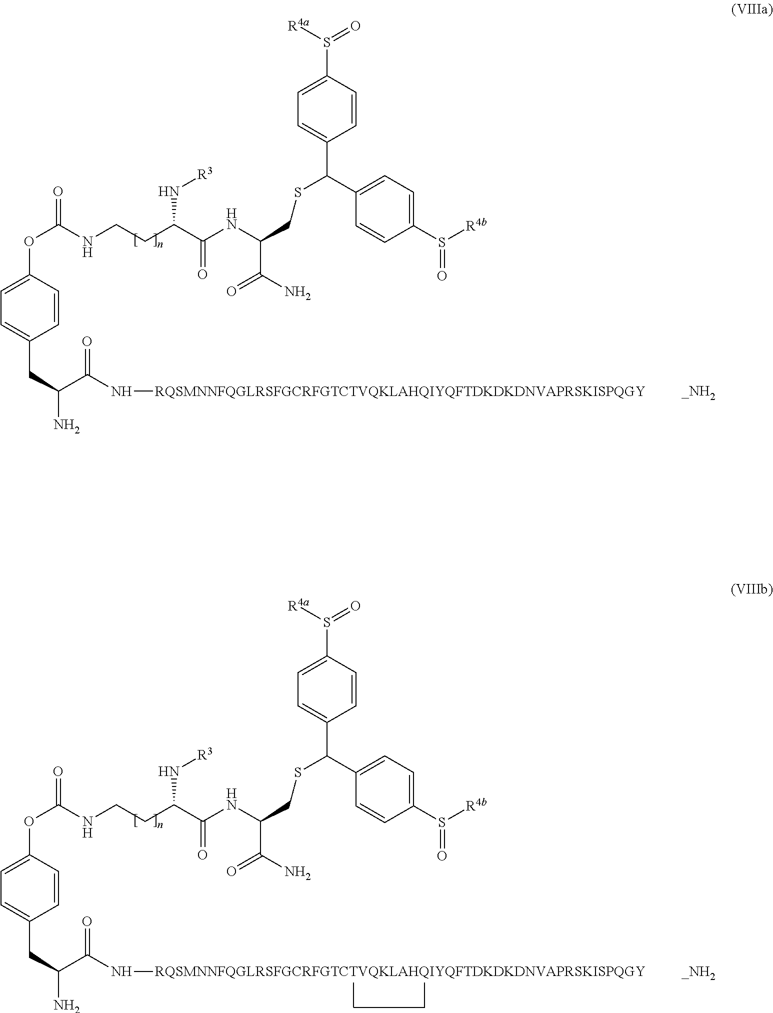
a hydrate thereof, solvate thereof, salt thereof, pharmaceutically acceptable salt thereof, or the solvates of salts thereof,
in which
n represents the number 1,
R1 represents an amine protecting group,
R2 represents an amine protecting group,
R3 represents hydrogen, methyl, ethyl, n-propyl or isopropyl,
R4a represents methyl, ethyl, propyl, isopropyl or butyl,
R4b represents methyl, ethyl, propyl, isopropyl or butyl,
R5 represents a linear or branched PEG 20 kDa to 80 kDa endcapped with a methoxy-group, wherein the amine protecting group is selected from Benzyloxycarbonyl (Cbz), p-Methoxybenzyl carbonyl (Moz or MeOZ), tert-butyloxycarbonyl (BOC), (9H-fluoren-9-ylmethoxy)carbonyl (FMOC), allyloxycarbonyl (Alloc), Carbamate group, p-Methoxybenzyl (PMB), 3,4-Dimethoxybenzyl (DMPM), p-Methoxyphenyl (PMP), Tosyl (Ts), Troc (trichloroethyl chloroformate) and sulfonamides (Nosyl, Nps).

12. A process for the preparation of the compound according to formula (IV), a hydrate thereof, solvate thereof, salt thereof, pharmaceutically acceptable salt thereof, or the solvates of salts thereof, wherein the process comprises step 1.1.b) according to claim 3.

13. A process for the preparation of the compound according to formula (VIIa) or (VIIb), wherein the process comprises a hydrate thereof, solvate thereof, salt thereof, pharmaceutically acceptable salt thereof, or the solvates of salts thereof, wherein the process comprises step 3) according to claim 1.

14. A process for the preparation of the compound according to formula (VIIIa), a hydrate thereof, solvate thereof, salt thereof, pharmaceutically acceptable salt thereof, or the solvates of salts thereof, wherein the process comprises step 4) according to claim 1.

15. A process for the preparation of the compound according to formula (VIIIb), a hydrate thereof, solvate thereof, salt thereof, pharmaceutically acceptable salt thereof, or the solvates of salts thereof, wherein the process comprises step 4) and/or step 5) according to claim 1.

16. The use of the compound according to any one of formulae (III-1), (IV), (VIIa), (VIIb), (VIIIa) and/or (VIIIb) in the process for the preparation of the compound according to formula (I) or (Ia).

17. The use of the compound according to any one of formulae (III-1), (IV), (VIIa) and/or (VIIb) in the process for the preparation of the compound according to formula (VIIIa) and/or (VIIIb).

18. The process according to claim 1, in which
n represents the number 1,
R¹ represents tert-butyloxycarbonyl or (9H-fluoren-9-ylmethoxy)carbonyl,
R² represents tert-butyloxycarbonyl,
R³ represents hydrogen, methyl,
R^{4a} represents methyl or ethyl,
R^{4b} represents methyl or ethyl,
R⁵ represents a linear or branched PEG 20 kDa to 80 kDa endcapped with a methoxy-group
a hydrate thereof, solvate thereof, salt thereof, pharmaceutically acceptable salt thereof, or the solvates of salts thereof.

19. The process according to claim 1, in which
n represents the number 1,
R¹ represents tert-butyloxycarbonyl or (9H-fluoren-9-ylmethoxy)carbonyl,
R² represents tert-butyloxycarbonyl,
R³ represents hydrogen, methyl or ethyl,
R^{4a} and R^{4b} represent methyl or ethyl,
R⁵ represents a linear or branched PEG 40 kDa endcapped with a methoxy-group,
a hydrate thereof, solvate thereof, salt thereof, pharmaceutically acceptable salt thereof, or the solvates of salts thereof.


SEQUENCE LISTING (2 sequences)

SEQ ID NO: 1 - protein, 51 aa, synthetic construct
  REGION 1..51: note = Adrenomedullin 2 - 52
  DISULFID 15..20: note = Intrachain
  MOD_RES 51: note = AMIDATION
RQSMNNFQGLRSFGCRFGTCTVQKLAHQIYQFTDKDKDNVAPRSKISPQGY

SEQ ID NO: 2 - protein, 52 aa, synthetic construct
  REGION 1..52: note = modified Adrenomeddulin 1 - 52
  MOD_RES 1: note = modified Tyrosine residue
  DISULFID 16..21: note = Intrachain
  MOD_RES 52: note = AMIDATION
YRQSMNNFQGLRSFGCRFGTCTVQKLAHQIYQFTDKDKDNVAPRSKISPQGY